(19) United States
(12) Patent Application Publication
(10) Pub. No.: US 2024/0002920 A1
(43) Pub. Date: Jan. 4, 2024

(54) METHOD AND KIT FOR DETECTING DNA METHYLATION BASED ON QUANTITATIVE POLYMERASE CHAIN REACTION (qPCR)

(71) Applicant: Yunfeng ZHU, Beijing (CN)

(72) Inventor: Yunfeng ZHU, Beijing (CN)

(21) Appl. No.: 18/252,844

(22) PCT Filed: Nov. 2, 2022
(86) PCT No.: PCT/CN2022/129141

§ 371 Date: May 12, 2023

(30) Foreign Application Priority Data
Nov. 5, 2021 (CN) 202111305138.9

(51) Int. Cl. C12Q 1/6851; C12Q 1/6886

(52) U.S. Cl. CPC C12Q 1/6851; C12Q 1/6886; C12Q 2600/154

(57) ABSTRACT

Provided is a method and kit for detecting DNA methylation based on quantitative polymerase chain reaction (qPCR). The present disclosure achieves the detection of DNA methylation based on qPCR, where PCR is used to prepare fragments of different DNA methylation levels through base incorporation, and then the fragments are subjected to qPCR detection with two programs respectively to get ΔCt and establish a correspondence between ΔCt and a DNA methylation level. The method evaluates a DNA methylation level of a target gene by detecting ΔCt of the target gene. The present disclosure establishes a new convenient method for detecting a DNA methylation difference. According to verification results of experiments with lung cancer and colorectal cancer plasma samples, the method has simple operations, short detection time, and accurate and reliable results, is suitable for the detection of all types of methylated DNA fragments, and has high application values.

### CROSS REFERENCE TO RELATED APPLICATION

The present application is a national stage application of International Patent Application No. PCT/CN2022/129141, filed on Nov. 2, 2022, which claims priority to Chinese Patent No. 202111305138.9 filed to the China National Intellectual Property Administration (CNIPA) on Nov. 5, 2021 and entitled “METHOD AND KIT FOR DETECTING DNA METHYLATION BASED ON QUANTITATIVE POLYMERASE CHAIN REACTION (qPCR)”, which is incorporated herein by reference in its entirety.

### REFERENCE TO SEQUENCE LISTING

A computer readable XML file entitled “GWPCTP20220902176.xml”, created on Apr. 17, 2023, with a file size of about 45,386 bytes, contains the sequence listing for this application, has been filed with this application, and is hereby incorporated by reference in its entirety.

### TECHNICAL FIELD

The present disclosure relates to the field of biotechnology, and in particular to a method and kit for detecting DNA methylation.

### BACKGROUND

Studies have shown that epigenetic modification of DNA plays an important role in gene regulation, and DNA methylation is a hotspot in epigenetic modification research. It is known that, during the occurrence and development of a tumor, the abnormal methylation of a gene is more abundant than structural changes of the gene (such as gene mutation, gene deletion, and gene fusion), and the gene methylation change-based detection has advantages over the gene structural change-based detection, which is attributed to the following reasons: the site and frequency of occurrence of a structural change of a gene have great uncertainty and are more likely to be sporadic; and DNA with an abnormal genetic structure accounts for a very low proportion in a tumor at an early stage, and thus it is very difficult to detect the DNA with an abnormal genetic structure in early of cancer. A gene methylation change occurs early and has high abundance, and thus the detection of the gene methylation change can greatly improve the sensitivity of determination. At present, the gene methylation-based detection has become the main means of epigenetic testing due to its strong operability, and is used in all aspects of molecular biology research. Therefore, the development of a new method for efficiently and sensitively detecting DNA methylation will undoubtedly greatly promote the progress of epigenetic research and molecular biology research.

Although there are currently many types of common DNA methylation detection methods, an underlying core technology of each of these DNA methylation detection methods is achieved based on bisulfite sodium treatment or methylation-sensitive restriction endonuclease treatment, where a treatment is first conducted by this technology and then a detection is conducted with a downstream test platform, such as MSP, HRM, and NGS. The use of bisulfite sodium treatment has the following problems: the insufficient transformation of a target base (a non-methylated cytosine nucleotide) brings artificial heterogeneity to subsequent detection, resulting in inaccurate results; the bisulfite sodium treatment will also lead to DNA breakage, which brings artificial uncertainty to subsequent detection and affects the determination of a result; and the analysis method also has disadvantages such as complicated procedures, too high cost, time-consuming, and high requirements for DNA quality and quantity. Due to the limitation of an specific site of a methylation-sensitive restriction endonuclease, the methylation-sensitive restriction endonuclease treatment technique cannot detect a region without the specific site, and thus the technique is not universal. Therefore, the establishment of a DNA methylation detection method with high specificity and high sensitivity has become an urgent need to break through the bottleneck of methylation detection.

### SUMMARY

### 1. Establishment of a Method for Detecting DNA Methylation:

The present disclosure is intended to provide a method and kit for detecting DNA methylation based on qPCR, which have the characteristics of simplicity, rapidity, and accurate and reliable results. The present disclosure provides a kit for detecting DNA methylation based on qPCR, including the following components: a Taq enzyme premix system (Taq enzyme, buffer, and dNTP), primers and probes of target genes for qPCR, a reagent for quality control of an activity of a Taq enzyme, reagents for plotting a reference curve and instructions for parameter set and procedure.

The present disclosure achieves the detection of DNA methylation based on qPCR, and a schematic map to account for detection mechanism is shown in FIG. 1, where a difference in DNA methylation is converted into a difference in effective PCR template amount based on the influence of methylation on physical and chemical properties of DNA, and then the difference can be detected by qPCR. That is, during a detection process, a sample is subjected to PCR amplification under both methylated DNA-insensitive conditions and methylated DNA-sensitive conditions. An amplification result (Ct value) obtained under methylation-insensitive conditions reflects an amount of template DNA, while an amplification result (Ct value) obtained under methylation-sensitive conditions reflects both an amount of template DNA and a methylation level of template DNA. Through self-control, a difference (ΔCt value) between the Ct values obtained under methylation-sensitive conditions and methylation-insensitive conditions is calculated to evaluate the template DNA methylation amount, and the ΔCt value is only related to the methylation level of the template DNA.

The kit of the present disclosure has no special limitations on an object to be tested, and is suitable for gene fragments of different methylation levels. In an embodiment of the present disclosure, the feasibility of the method is verified using APC, SHOX2, Apobec3B, and P53 gene fragments.

Preferably, the reagents for plotting the reference curve include deoxynucleotide triphosphate (dNTP) reagents with normal dATP, dGTP, dTTP and mixture of dCTP with different proportions of methylated cytosine (5′-m-dCTP) as well as another reagent for polymerase chain reaction (PCR).

Preferably, the dNTP reagents with different proportions of methylated cytosine each include equal molar concentrations of deoxyadenosine triphosphate (dATP), deoxythymidine triphosphate (dTTP), deoxyguanosine triphosphate (dGTP), and a reagent A; and
the reagent A is 5′-methyl-deoxycytidine triphosphate (5′-m-dCTP) and/or deoxycytidine triphosphate (dCTP).

In the reagent A, a molar ratio of the 5′-m-dCTP to the dCTP falls into the parameters as follows: 1:0, 1:(1-30), and 0:1.

The kit provided in the present disclosure achieves the detection of DNA methylation based on qPCR, where PCR is used to prepare fragments of different DNA methylation levels through base incorporation, and then the fragments are subjected to qPCR detection to establish a relationship between ΔCt and a DNA methylation level, that is, the higher the DNA methylation level, the larger the ΔCt. Therefore, the kit realizes the evaluation of a DNA methylation level of a target gene by detecting ΔCt of the target gene. The method has simple operations, short detection time, and accurate and reliable results, is suitable for the detection of all types of methylated DNA fragments, and has high application values.

The present disclosure provides a method for detecting DNA methylation, including the following steps:
1) using the primers and probes in the kit to prepare a reaction system for qPCR detection, and conducting high-temperature denaturation amplification and low-temperature denaturation amplification respectively, with which Ct_{X-HT} accounts for X gene in high-temperature denaturation amplification and Ct_{X-LH} accounts for X gene in low-temperature denaturation amplification;
2) substituting _{X-LH} and Ct_{X-HT} obtained in step 1) into equation Ito obtain ΔCtx of a X gene,

ΔCtx=Ct_{X-LH}−Ct_{X-HT} equation I
where Ct_{X-LH} represents a Ct value of an X gene when amplified under low-temperature denaturation, Ct_{X-HT} represents a Ct value of an X gene when amplified under high-temperature denaturation, and X represents a target gene;
3) using the reagents for plotting the reference curve in the kit by amplifying templates with different DNA methylation levels prepared prior by PCR with 5′-m-dCTP incorporation . Subjecting resulting PCR products to the operations in step 1) and step 2) to obtain ΔCtx′ of the fragments of different DNA methylation levels, and based on a logarithm relationship between different proportions of methylated cytosine and ΔCtx′, plotting the reference curve; and
4) comparing ACtx of the target gene in step 2) with ΔCtx′ in the reference curve in step 3) to obtain a proportion of methylated cytosine in the target gene, such as to determine a DNA methylation level of the target gene.

Reaction conditions for the high-temperature denaturation amplification vary depending on a gene sequence of a target region to be amplified and are preferably as follows: predenaturation at 95° C. for 5 min; and 94° C. for 5 s, 60° C. to 62° C. for 15 s, and 72° C. for 30 s, with 45 cycles; and
reaction conditions for the low-temperature denaturation amplification vary depending on a gene sequence of a target region to be amplified and are preferably as follows: 85° C. to 88° C. for s, 60° C. to 62° C. for 15 s, and 72° C. for 30 s, with 18 cycles; and 94° C. for 5 s, 60° C. to 62° C. for s, and 72° C. for 30 s, with 27 cycles.
   2. Establishment of a Method for Combined Analysis of a Methylation Difference of a Target Gene between Two Populations:

The present disclosure provides a use of the kit or the method according to the above technical solution in distinguishing a DNA methylation change of one study population from a DNA methylation change of the other study population.

When the target gene includes two or more target genes, a method for distinguishing a DNA methylation change of one study population from a DNA methylation change of the other study population includes the following steps:
after ΔCt of each target gene is obtained, subjecting ΔCt of a target gene corresponding to each of samples of a population 1 and samples of a population 2 to statistical analysis to obtain a weight value equation II and a threshold for determining a difference between the two populations, where ΔCt of each target gene is substituted into the weight value equation II to calculate a weight value for joint detection of multiple target genes of each sample,

weight value for joint detection of multiple target genes=a1×ΔCtₓ₁+a2×ΔCtₓ₂+. . . +a2×ΔCtₓₙ equation II
where “a1”, “a2”, and “an” each represent a corresponding coefficient obtained during statistical analysis of each target gene;
qualitative comparison between the two populations: comparing a weight value of each of the samples in the population 1 and the samples in the population 2 with a threshold, where a sample with a weight value lower the threshold is a lowly methylated sample, and a sample with a weight value higher than the threshold is a highly methylated sample; and determining a difference between the two populations through statistical analysis; and
differential determination between the two populations: calculating a mean value of weight values of the samples in the population 1 and a mean value of weight values of the samples in the population 2, and conducting T-test analysis, where when the mean value of the weight values of the samples in the population 1 is significantly different from the mean value of the weight values of the samples in the population 2, it indicates that there is a significant DNA methylation difference between the two populations.

The statistical analysis includes binary regression analysis, T-test analysis, or the like.

### 3. The Target Gene of the Present Disclosure Includes: APC, SHOX2, Apobec3B, P53, AID, HOXD12, Alu, SDC2, WDR17, and ADHFE1.

Primer sequences for qPCR of APC are an upstream primer sequence shown in SEQ ID NO: 1 and a downstream primer sequence shown in SEQ ID NO: 2; and a probe for qPCR of APC is a nucleotide sequence shown in SEQ ID NO: 3.

Primer sequences for qPCR of SHOX2 are an upstream primer sequence shown in SEQ ID NO: 4 and a downstream primer sequence shown in SEQ ID NO: 5; and a probe sequence for qPCR of SHOX2 is a sequence shown in SEQ ID NO: 6.

Primer sequences for qPCR of Apobec3B are an upstream primer sequence shown in SEQ ID NO: 7 and a downstream primer sequence shown in SEQ ID NO: 8; and a probe sequence for qPCR of Apobec3B is a sequence shown in SEQ ID NO: 9.

Primer sequences for qPCR of P53 are an upstream primer sequence shown in SEQ ID NO: 10 and a downstream primer sequence shown in SEQ ID NO: 11; and a probe sequence for qPCR of P53 is a sequence shown in SEQ ID NO: 12.

Primer sequences for qPCR of AID are an upstream primer sequence shown in SEQ ID NO: 13 and a downstream primer sequence shown in SEQ ID NO: 14; and a probe sequence for qPCR of AID is a sequence shown in SEQ ID NO: 15.

Primer sequences for qPCR of HOXD12 are an upstream primer sequence shown in SEQ ID NO: 16 and a downstream primer sequence shown in SEQ ID NO: 17; and a probe sequence for qPCR of HOXD12 is a sequence shown in SEQ ID NO: 18.

Primer sequences for qPCR of Alu are an upstream primer sequence shown in SEQ ID NO: 19 and a downstream primer sequence shown in SEQ ID NO: 20; and a probe sequence for qPCR of Alu is a sequence shown in SEQ ID NO: 21.

Primer sequences for qPCR of SDC2 are an upstream primer sequence shown in SEQ ID NO: 22 and a downstream primer sequence shown in SEQ ID NO: 23; and a probe sequence for qPCR of SDC2 is a sequence shown in SEQ ID NO: 24.

Primer sequences for qPCR of WDR17 are an upstream primer sequence shown in SEQ ID NO: 25 and a downstream primer sequence shown in SEQ ID NO: 26; and a probe sequence for qPCR of WDR17 is a sequence shown in SEQ ID NO: 27.

Primer sequences for qPCR of ADHFE1 are an upstream primer sequence shown in SEQ ID NO: 28 and a downstream primer sequence shown in SEQ ID NO: 29; and a probe sequence for qPCR of ADHFE1 is a sequence shown in SEQ ID NO: 30.

### BRIEF DESCRIPTION OF THE DRAWINGS

FIG. 1 shows the unwinding differences of different proportions of methylated DNA under low-temperature denaturation;

FIG. 2 shows a ΔCt change curve of SHOX2 gene fragments with different methylation levels, where the abscissa represents a molar ratio of 5′-m-dCTP to dCTP and the ordinate represents ΔCt;

FIG. 3 is a fitted logarithmic curve illustrating a relationship between methylation and detection value of an SHOX2 gene; where the abscissa represents ΔCt and the ordinate represents a molar ratio of 5′-m-dCTP to dCTP;

FIG. 4 shows a ΔCt change curve of P53 gene fragments with different methylation levels, where the abscissa represents a molar ratio of 5′-m-dCTP to dCTP and the ordinate represents ΔCt;

FIG. 5 is a fitted logarithmic curve illustrating a relationship between methylation and detection value of a P53 gene; where the abscissa represents ΔCt and the ordinate represents a molar ratio of 5′-m-dCTP to dCTP;

FIG. 6 shows a ΔCt change curve of APC gene fragments with different methylation levels, where the abscissa represents a molar ratio of 5′-m-dCTP to dCTP and the ordinate represents ΔCt;

FIG. 7 is a fitted logarithmic curve illustrating a relationship between methylation and detection value of an APC gene; where the abscissa represents ΔCt and the ordinate represents a molar ratio of 5′-m-dCTP to dCTP;

FIG. 8 shows a ΔCt change curve of Apobec3B gene fragments with different methylation levels, where the abscissa represents a molar ratio of 5′-m-dCTP to dCTP and the ordinate represents ΔCt;

FIG. 9 is a fitted logarithmic curve illustrating a relationship between methylation and detection value of a Apobec3B gene; where the abscissa represents ΔCt and the ordinate represents a molar ratio of 5′-m-dCTP to dCTP;

FIG. 10 shows a ΔCt change curve of AID gene fragments with different methylation levels, where the abscissa represents a molar ratio of 5′-m-dCTP to dCTP and the ordinate represents ΔCt;

FIG. 11 is a fitted logarithmic curve illustrating a relationship between methylation and detection value of an AID gene; where the abscissa represents ΔCt and the ordinate represents a molar ratio of 5′-m-dCTP to dCTP;

FIG. 12 shows a A Ct change curve of HOXD12 gene fragments with different methylation levels, where the abscissa represents a molar ratio of 5′-m-dCTP to dCTP and the ordinate represents ΔCt;

FIG. 13 is a fitted logarithmic curve illustrating a relationship between methylation and detection value of an HOXD12 gene; where the abscissa represents ΔCt and the ordinate represents a molar ratio of 5′-m-dCTP to dCTP;

FIGS. 14A-B show the analysis of test results of lung cancer samples and healthy samples, where FIG. 14A is a box plot and FIG. 14B is a receiver-operating characteristic (ROC) curve;

FIG. 15 shows a ΔCt change curve of SDC2 gene fragments with different methylation levels, where the abscissa represents a molar ratio of 5′-m-dCTP to dCTP and the ordinate represents ΔCt;

FIG. 16 is a fitted logarithmic curve illustrating a relationship between methylation and detection value of an SDC2 gene; where the abscissa represents ΔCt and the ordinate represents a molar ratio of 5′-m-dCTP to dCTP;

FIG. 17 shows a ΔCt change curve of WDR17 gene fragments with different methylation levels, where the abscissa represents a molar ratio of 5′-m-dCTP to dCTP and the ordinate represents ΔCt;

FIG. 18 is a fitted logarithmic curve illustrating a relationship between methylation and detection value of a WDR17 gene; where the abscissa represents ΔCt and the ordinate represents a molar ratio of 5′-m-dCTP to dCTP;

FIG. 19 shows a ΔCt change curve of ADHFE1 gene fragments with different methylation levels, where the abscissa represents a molar ratio of 5′-m-dCTP to dCTP and the ordinate represents ΔCt;

FIG. 20 is a fitted logarithmic curve illustrating a relationship between methylation and detection value of an ADHFE1 gene; where the abscissa represents ΔCt and the ordinate represents a molar ratio of 5′-m-dCTP to dCTP; and

FIGS. 21A-B show the analysis of test results of CRC samples and healthy samples, where FIG. 21A is a box plot and FIG. 21B is an ROC curve.

### DETAILED DESCRIPTION OF THE EMBODIMENTS

### 1. Establishment of a Method for Detecting DNA Methylation:

The present disclosure provides a kit for detecting DNA methylation based on qPCR, including the parameter set for methylation-insensitive and methylation-sensitive conditions, relative formula as well as following components: a Taq enzyme premix system, primers and probes of target genes for qPCR, and a reagent for quality control of an activity of a Taq enzyme.

The kit of the present disclosure has no special limitations on an object to be tested, and is suitable for gene fragments of different methylation levels. In an embodiment of the present disclosure, the feasibility of the kit and method is verified using APC, SHOX2, Apobec3B, and P53.

In an embodiment of the present disclosure, with APC gene fragments as an example, the method for detecting DNA methylation is verified: with plasma cell-free DNA (cfDNA) as a template, an APC gene is prepared through PCR amplification, and the specificity of a primer pair is verified; a PCR product of the APC gene is diluted and used as a template to conduct PCR amplification, during which methylated cytosine incorporation is conducted with dNTP including dATP/dGTP/dTTP and a mixture of different ratios of 5′-m-dCTP to dCTP to prepare DNA fragments with different methylation levels; and methylated DNA is used as a template to conduct qPCR detection under two conditions (high temperature and low temperature), and a difference between two resulting Ct values is calculated to obtain a ΔCt value.

It is confirmed that the ΔCt value is only correlated with a methylation level of DNA and is not correlated with a concentration of DNA, and the ΔCt value is positively correlated with the methylation level of DNA. Primer sequences for qPCR of APC are an upstream primer sequence shown in SEQ ID NO: 1 and a downstream primer sequence shown in SEQ ID NO: 2; and a probe for qPCR of APC is a nucleotide sequence shown in SEQ ID NO: 3.

A reference curve for detecting DNA methylation: DNA fragments with different methylation levels each are compared with a corresponding non-methylated DNA fragment, and it is found that different gene fragments correspond to different methylation detection limit values, such that different cytosine proportions need to be set for different types of target genes when a reference curve is plotted. ΔCtx of a target gene is compared with ΔCtx′ in the reference curve to obtain a proportion of methylated cytosine in the target gene, such as to determine a DNA methylation level of the target gene. In this way, a DNA methylation level of each target gene can be quantitatively evaluated through the reference curve.

The methylation level of the target gene is determined using dNTP reagents with different proportions of methylated cytosine. The dNTP reagents with different proportions of methylated cytosine each preferably include equal molar concentrations of dATP, dTTP, dGTP, and a reagent A. Preferably, the concentrations of the dATP, dTTP, dGTP, and reagent A each are independently 2.5 mM. The reagent A is 5′-m-dCTP and/or dCTP. In the reagent A, a molar ratio of 5′-m-dCTP to dCTP is preferably as follows: 1:0, 1:(1-30), or 0:1, where 1:(1-30) includes multiple ratio values of 1:1, 1:2, 1:3 . . . 1:29, and 1:30.
1) Preparation of a PCR mixture for DNA with different methylation levels:

| Component | | Volume (μL) |
|---|---|---|
| PCR solution | Taq enzyme (5 U/μL) | 0.5 |
| | Upstream primer for PCR | 0.5 |
| | Downstream primer for PCR | 0.5 |
| | 10 × PCR buffer | 2 |
| | dNTP (dATP, dTTP, dGTP, and reagent A) | 2 |

| | |
|---|---|
| DNA template for target gene (PCR product diluted by 10⁶) | 2 |

A reaction system is 20 μL; and if the volume is insufficient, ddH₂O is added to the volume.

2) Preparation of a aPCR mixture for DNA methvlation detection:

| Component | | Volume (μL) |
|---|---|---|
| qPCR solution | Taq enzyme premix system (2 × Premix | 10 |
| | Ex Taq ™) | |
| | Upstream primer for PCR | 0.5 |
| | Downstream primer for PCR | 0.5 |
| | Taqman probe | 0.25 |

| | |
|---|---|
| Methylated DNA template prepared above (PCR product | 2 |
| diluted by 10⁶) | |

A reaction system is 20 μL; and if the volume is insufficient, ddH₂O is added to the volume.

3) Use of the kit provided by the present disclosure in the detection of DNA methylation

The present disclosure provides a method for detecting DNA methylation, including the following steps:
(1) the primers and probes in the kit are used to prepare a reaction system for qPCR detection, and high-temperature denaturation amplification and low-temperature denaturation amplification are conducted to obtain Ct_{X-HT} for the high-temperature denaturation amplification and Ct_{X-LH} for the low-temperature denaturation amplification respectively;
(2) Ct_{X-LH} and Ct_{X-HT} obtained in step (1) are substituted into equation I to obtain ΔCtₓ of a target gene,

ΔCtₓ=Ct_{X-LH}−Ct_{X-HT} equation I

where Ct_{X-LH} represents a Ct value of an X gene when amplified under low-temperature denaturation, Ct_{X-HT} represents a Ct value of an X gene when amplified under high-temperature denaturation, and X represents a target gene;
(3) the reagents for plotting the reference curve in the kit are used to amplify target gene fragments to prepare different DNA methylation levels, resulting PCR data are subjected to the operations in step (1) and step (2) to obtain ΔCtx′ of the fragments of different DNA methylation levels, and based on a logarithm relationship between different proportions of methylated cytosine and ΔCtx′, the reference curve is plotted; and
(4) ΔCtₓ of the target gene in step (2) is compared with ACtx′ in the reference curve in step (3) to obtain a proportion of methylated cytosine in the target gene, such as to determine a DNA methylation level of the target gene.

Reaction conditions for the high-temperature denaturation amplification vary depending on a sequence of a target gene to be amplified and are preferably as follows: predenaturation at for 5 min; and 94° C. for 5 s, 60° C. to 62° C. for 15 s, and 72° C. for 30 s, with 45 cycles; and
reaction conditions for the low-temperature denaturation amplification vary depending on a sequence of a target gene to be amplified and are preferably as follows: 85° C. to 88° C. for 15 s, to 62° C. for 15 s, and 72° C. for 30 s, with 18 cycles; and 94° C. for 5 s, 60° C. to 62° C. for 15 s, and 72° C. for 30 s, with 27 cycles.
   2. Establishment of a Method for Combined Analysis of a Methylation Difference of a Target Gene between Two Populations:

In the present disclosure, a use of the method for detecting DNA methylation described in the technical solution for detection of lung cancer and Colorectal Cancer (CRC) is explored, and the kit can also be used in the detection of DNA methylation in any tumor sample as well as other disease, where DNA is a target gene fragment of free DNA in peripheral blood as well as genomic DNA in cells.

A method for distinguishing a DNA methylation change of one study population from a DNA methylation change of the other study population includes the following steps:
after ΔCt of each target gene is obtained, ΔCt of a target gene corresponding to each of samples of a population 1 and samples of a population 2 is subjected to binary regression statistical analysis to obtain a weight value equation II and a threshold for determining a difference between the two populations, where ΔCt of each target gene is substituted into the weight value equation II to calculate a weight value for joint detection of multiple target genes of each sample,

weight value for joint detection of multiple target genes=a1×ΔCt_{X1}+a2×ΔCt_{X2}+. . . +an×ΔCtₓₙ equation II
where “a1”, “a2”, and “an” each represent a corresponding coefficient obtained during statistical analysis of each target gene;
qualitative comparison between the two populations: a weight value of each of the samples in the population 1 and the samples in the population 2 is compared with the threshold, where a sample with a weight value lower than the threshold is a lowly methylated sample and a sample with a weight value higher than the threshold is a highly methylated sample; and a difference between the two populations is determined through statistical analysis; and
differential determination between the two populations: a mean value of weight values of the samples in the population 1 and a mean value of weight values of the samples in the population 2 each are calculated, and T test analysis is conducted, where when the mean value of the weight values of the samples in the population 1 is significantly different from the mean value of the weight values of the samples in the population 2, it indicates that there is a significant DNA methylation difference between the two populations.
(1) In an embodiment of the present disclosure, when a lung cancer sample is subjected to detection for identification, the target genes for DNA methylation detection are AID and HOXD12; the target gene for DNA amount detection in peripheral blood of a lung cancer is Alu, which is a high-copy repeat sequence in a human genome. In this process, primer design software is used to conduct primer design for a target gene, in which the fitted CG amount in target region is considered for methyaltion detection, the specificity of different candidate primers are evaluated by amplifying the target gene with template DNA. The primers for amplification of specific bands are selected as primers for qPCR. Primer sequences for qPCR of AID are an upstream primer sequence shown in SEQ ID NO: 13 and a downstream primer sequence shown in SEQ ID NO: 14, and a probe sequence for qPCR of AID is a sequence shown in SEQ ID NO: 15. Primer sequences for qPCR of HOXD12 are an upstream primer sequence shown in SEQ ID NO: 16 and a downstream primer sequence shown in SEQ ID NO: 17, and a probe sequence for qPCR of HOXD12 is a sequence shown in SEQ ID NO: 18. Primer sequences for qPCR of Alu are an upstream primer sequence shown in SEQ ID NO: 19 and a downstream primer sequence shown in SEQ ID NO: 20, and a probe sequence for qPCR of Alu is a sequence shown in SEQ ID NO: 21.

Reference curve analysis of a methylation level of a target gene: When AID is subjected to DNA methylation detection, the plotting of a reference curve requires the preparation of a reagent A prior in the following molar ratio of 5′-m-dCTP to dCTP: 1:0, 1:1, 1:2, 1:3, 1:4, 1:5, 1:6, and 0:1. When HOXD12 is subjected to DNA methylatation detection, the plotting of a reference curve requires the preparation of a reagent A prior in the following molar ratio of 5′-m-dCTP to dCTP: 1:0, 1:1, 1:2, 1:3, 1:4, 1:5, 1:6, 1:8, 1:10, 1:20, and 0:1.
(2) In an embodiment of the present disclosure, when a CRC sample is subjected to detection for identification, The target genes for DNA methylation detection are SDC2, WDR17, and ADHFE1; the target gene for DNA amount detection in peripheral blood of CRC is Alu, which is a high-copy repeat sequence in human genome. In this process, primer design software is used to conduct primer design for a target gene, in which the fitted CG amount in target region is considered for methyaltion detection. The specificity of different candidate primers are evaluated by amplifying the target gene with template DNA. The primers for amplification of specific bands are selected as primers for qPCR.

Primer and probe sequences for qPCR of a target gene: Primer sequences for qPCR of Alu are an upstream primer sequence shown in SEQ ID NO: 19 and a downstream primer sequence shown in SEQ ID NO: 20, and a probe sequence for qPCR of Alu is a sequence shown in SEQ ID NO: 21. Primer sequences for qPCR of SDC2 are an upstream primer sequence shown in SEQ ID NO: 22 and a downstream primer sequence shown in SEQ ID NO: 23, and a probe sequence for qPCR of SDC2 is a sequence shown in SEQ ID NO: 24. Primer sequences for qPCR of WDR17 are an upstream primer sequence shown in SEQ ID NO: 25 and a downstream primer sequence shown in SEQ ID NO: 26, and a probe sequence for qPCR of WDR17 is a sequence shown in SEQ ID NO: 27. Primer sequences for qPCR of ADHFE1 are an upstream primer sequence shown in SEQ ID NO: 28 and a downstream primer sequence shown in SEQ ID NO: 29, and a probe sequence for qPCR of ADHFE1 is a sequence shown in SEQ ID NO: 30.

Reference curve analysis of a methylation level of a target gene: When SDC2 is subjected to DNA methylatation detection, the plotting of a reference curve requires the preparation of a reagent A prior in the following molar ratio of 5′-m-dCTP to dCTP: 1:0, 1:1, 1:2, 1:4, 1:6, 1:8, 1:10, and 0:1. When WDR17 is subjected to DNA methylatation detection, the plotting of a reference curve requires the preparation of a reagent A prior in the following molar ratio of 5′-m-dCTP to dCTP: 1:0, 1:1, 1:2, 1:4, 1:6, 1:8, 1:10, 1:20, 1:30, and 0:1. When ADHFE1 is subjected to DNA methylatation detection, the plotting of a reference curve requires the preparation of a reagent A prior in the following molar ratio of 5′-m-dCTP to dCTP: 1:0, 1:1, 1:2, 1:4, 1:6, 1:8, 1:10, and 0:1.

The method and kit for detecting DNA methylation based on qPCR provided by the present disclosure will be described below in detail with reference to examples, but the examples should not be construed as limiting the protection scope of the present disclosure.

### Example 1

### Establishment of a Method for Detecting Methylation:

The study in this example showed that the physical and chemical properties of methylated DNA were significantly changed compared with non-methylated DNA, and based on this phenomenon, a new methylation detection method was developed. A basic principle was as follows: A difference in DNA methylation was converted into a difference in effective template amount for PCR under specified conditions, with which methylated DNA was amplified by qPCR under both methylation-sensitivive conditions and methylation-insensitive conditions, and a difference between two Ct values was calculated. During a modeling process, PCR was used to prepare methylated DNA fragments with different methylation levels through base incorporation, then a methylation difference was detected, and a correspondence between a detection value (ΔCt) and a methylation level was established. A specific method was as follows:
1) Experimental materials

Reagents: a Taq enzyme premix system (2×Premix Ex Taq™) and a Taq™ reagent were purchased from TaKaRa Bio Inc; 5′-m-dCTP (5′-methylcytosine) was purchased from New England Biolabs; a T/A carrier for target gene cloning was purchased from TIANGEN (Beijing) Biotech Co., Ltd.; and the primers and probes were synthesized by Sangon Biotech (Shanghai) Co., Ltd.

Target genes: During the exploration of the new detection method, the DNA fragment available in our laboratory were selected, which are promoter region of genes APC, Apobec3B, and SHOX2 and exon region of P53 (exon 4) fragments. The PCR primers of these target genes were designed and synthesized following by PCR amplification to verify the specificity of each primer pair
2) Preparation of methylated DNA fragments (PCR incorporation method): During the preparation of methylated DNA fragments, the dNTP including regent A (different of molar ratios:5′-m-dCTP to dCTP) and dATP/dTTP/dGTP was prepared prior, and PCR amplification was implemented with the templates of target genes. In this way, the preparation of target genes with different methylation level was completed.
3) PCR: Composition of a reaction mixture as well as reaction conditions.
   (1) Preparation of Templates with Different DNA Methylation Levels

Preparation of dNTP with different proportions of methylated cytosine (as shown in Table 1): 5′-m-dCTP and dCTP were first mixed in molar ratios of 1:0, 1:(1-30), and 0:1 respectively, and resulting mixtures each were further mixed with equal proportions of dATP, dTTP, and dGTP to complete the preparation of dNTP including different proportions of methylated cytosine (each base had a concentration of 2.5 mM). Primer sequences were shown in Table 3.

TABLE 1
PCR mixture (20 μL reaction system) for
the preparation of methylated DNA fragments

| | Component | Volume (μL) |
|---|---|---|
| PCR solution | dNTP (dATP, dTTP, dGTP, and reagent A) | 2 |
| | Upstream primer | 0.5 |
| | Downstream primer | 0.5 |
| | DNA template (target gene) | 2 |
| | 10 × Buffer | 2 |
| | Taq enzyme | 0.5 |

The reaction system is 20 μL; and if the volume is insufficient, ddH₂O is added to the volume.

PCR conditions (20 μL reaction system, as shown in Table 2): predenaturation at 95° C. for 5 min; and then 3-step amplification: 94° C. for 5 s, 60° C. for 15 s, and 72° C. for 30 s, with 45 cycles.
(2)

TABLE 2
Preparation of a qPCR mixture for
detecting methylated DNA fragments:

| Component | | Volume (μL) |
|---|---|---|
| qPCR solution | 2 × Premix Ex Taq ™ | 10 |
| | Upstream primer for PCR | 0.5 |
| | Downstream primer for PCR | 0.5 |
| | Taqman probe | 0.25 |

| | |
|---|---|
| Methylated DNA template (PCR product in Table 1 | 2 |
| diluted by 10⁶) | |

The reaction system is 20 μL; and if the volume is insufficient, ddH₂O is added to the volume.

TABLE 3
Primer and probe sequences

| | Upstream primer (5′-3′) | Downstream primer (5′-3′) | Probe (5′FAM-3′BHQ1) |
|---|---|---|---|
| APC | atcttgtgctaatccttctgc | ccgcacagcctgcctag | ctgcggacctcccccgactct |
| | (SEQ ID NO: 1) | (SEQ ID NO: 2) | (SEQ ID NO: 3) |
| SHOX2 | agttgcgaggggaggtaat | gggaaatgggaactgatgc | ccctgccggagcgcgcttgttc |
| | (SEQ ID NO: 4) | (SEQ ID NO: 5) | (SEQ ID NO: 6) |
| Apobec3B | gccagagccagagaaacatgaag | tgcttacagcgtccttgcagttg | ccggggcctcccacaccaatg |
| | (SEQ ID NO: 7) | (SEQ ID NO: 8) | (SEQ ID NO: 9) |
| P53 | cacttgtgccctgactttcaac | gtcatgtgctgtgactgcttg | agatggccatggcgcggacgcgg |
| | (SEQ ID NO: 10) | (SEQ ID NO: 11) | g (SEQ ID NO: 12) |

Notes: An amplified fragment of a target region of APC (promoter) has a length of 148 bp (5′-atcttgtgctaatccttctgccctgeggacctcccccgactctttactatgcgtgtcaactgccatcaacttccttgcttgctggggactggggcc gcgagggcatacccccgaggggtacggggctagggctaggcaggctgtgcgg-3′, SEQ ID NO: 31); an amplified fragment of a target region of SHOX2 (promoter) has a length of 174 bp (5′-agttgcgaggggaggtaatttactcaaaacaagaggccgccgttaggaagtataaatagtgggacttcaagcgcagcgtcgctatcagatc tgaactctccgcaggaagaattgtcaaagatcttaacattgaacaagcgcgctccggcagggaagcatcagttcccatttccc-3′, SEQ ID NO: 32); an amplified fragment of a target region of Apobec3B (promoter) has a length of 138 bp (5′-gccagagccagagaaacatgaagcaccccggggcctcccacaccaatgcctgagcaggaatggggaggggccatgactcataaggcc ctgggaggtcactttaaggagggctgtccaactgcaaggacgctgtaagca-3′, SEQ ID NO: 33); and an amplified fragment of a target region of P53 (exon 4) has a length of 180 bp (5′-cacttgtgccctgactttcaactctgtctccttcctc ttcctacagtactcccctgccctcaacaagatgttttgccaactggccaagacctgccctgtgcagctgtgggttgattccacacccccgccc ggcacccgcgtccgcgccatggccatctacaagcagtcacagcacatgac-3′, SEQ ID NO: 34)

PCR instrument: Hongshi SLAN 96P.

### Reaction Conditions:

(1) High-temperature denaturation (HT): 4 genes (APC/Apobec3B/P53/SHOX2): predenaturation at 95° C. for 5 min; and then 3-step amplification: 94° C. for 5 s, 60° C. for 15 s, and 72° C. for 30 s, with 45 cycles.
(2) Low-temperature denaturation* (LT): 4 genes (APC/Apobec3B/P53/SHOX2): 85° C. for 15 s, 60° C. for 15 s, and 72° C. for 30 s, with 18 cycles; and then the following cyclic program: 94° C. for 5 s, 60° C. for 15 s, and 72° C. for 30 s, with 27 cycles.

Appropriate adjustments could be made according to a length of an amplified fragment and a CG content.
(3) Calculation of ΔCt

After data in PCR results were exported, a corresponding value was calculated according to equation I below.

ΔCtₓ=Ct_{X-LH}−Ct_{X-HT} equation I
where Ct_{X-LH} represents a Ct value of an X gene when amplified under low-temperature denaturation and Ct_{X-HT} represents a Ct value of an X gene when amplified under high-temperature denaturation.
4) Experimental results
(1) ΔCt was an independent parameter regardless of template concentration: Different methylated DNA fragments prepared each were subjected to gradient dilution and then used as a template to conduct qPCR analysis under two temperature conditions, where Ct_{X-HT} was defined as Ct1, Ct_{X-LH} was defined as Ct2, and a difference between Ct2 and Ct1 was defined as ΔCt.

Test results of promoter fragments of different target genes were as follows:

Test results of SHOX2 gene fragments were shown in Table 4.

TABLE 4
Test analysis of SHOX2 gene fragments by qPCR with different
template concentrations and different methylation levels

| | Methylated DNA fragment (reagent A: 5′-m-dCTP:dCTP) | | | | | |
|---|---|---|---|---|---|---|
| | 0:1 | 0:1 | 1:0 | 1:0 | 1:1 | 1:1 |
| Dilution ratio | 10⁵ | 10⁶ | 10⁵ | 10⁶ | 10⁵ | 10⁶ |
| Ct2 | 16.78 | 20.25 | 19.75 | 23.7 | 17.97 | 21.55 |
| Ct1 | 16.72 | 20.37 | 16.46 | 20.27 | 16.23 | 19.81 |
| ΔCt | 0.06 | −0.12 | 3.29 | 3.43 | 1.74 | 1.74 |

The results showed that, although the Ct values (Ct1 and Ct2) changed accordingly at different template concentrations, the ΔCt value remained almost unchanged; and the ΔCt values at different methylation levels were significantly different, indicating that the ΔCt value was only correlated with a methylation level of DNA and was not correlated with a concentration of DNA template, and the ΔCt value was positively correlated with the methylation level of DNA.
(2) ΔCt was positively correlated with a methylation level of a template

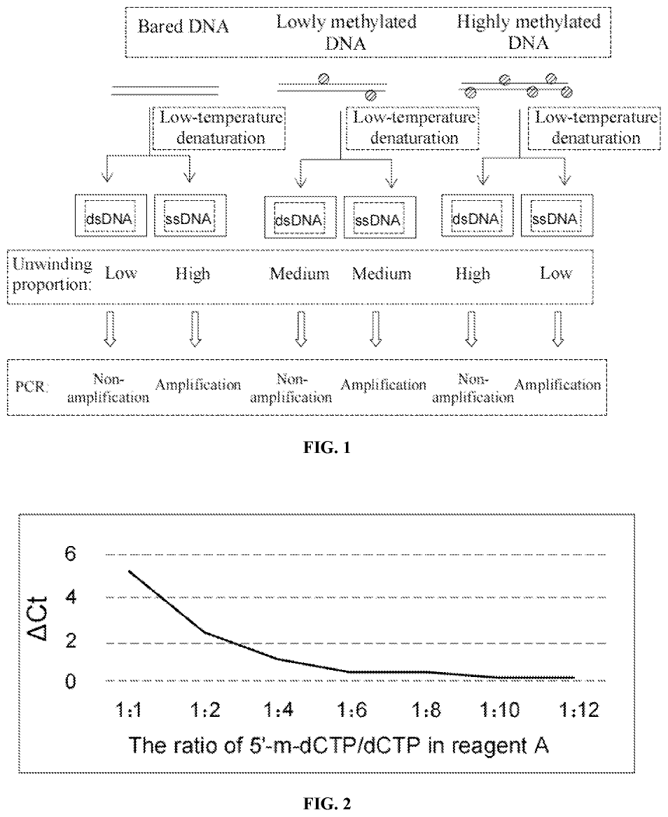

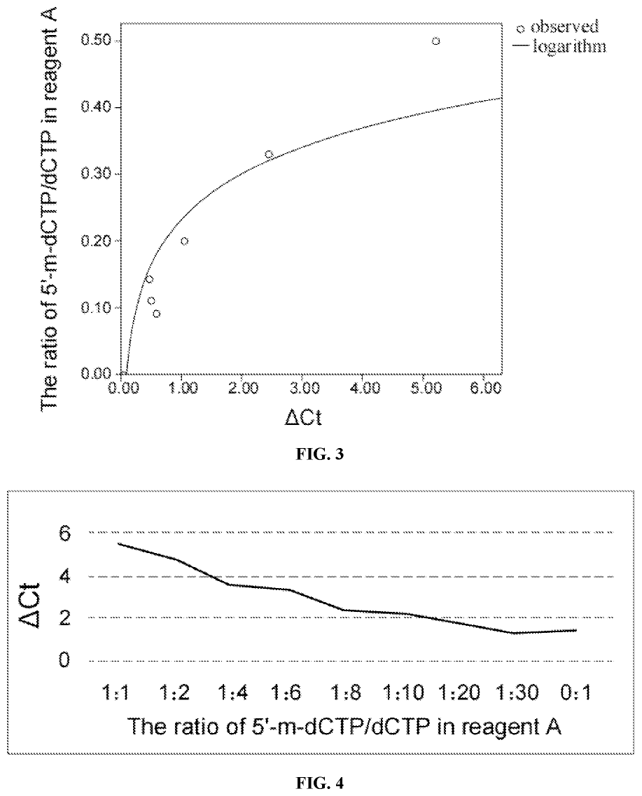

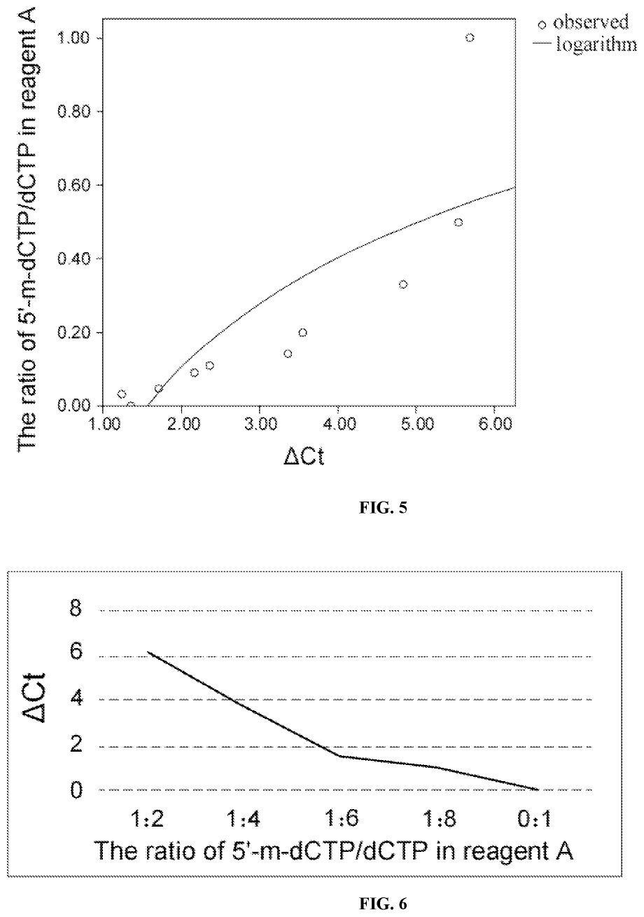

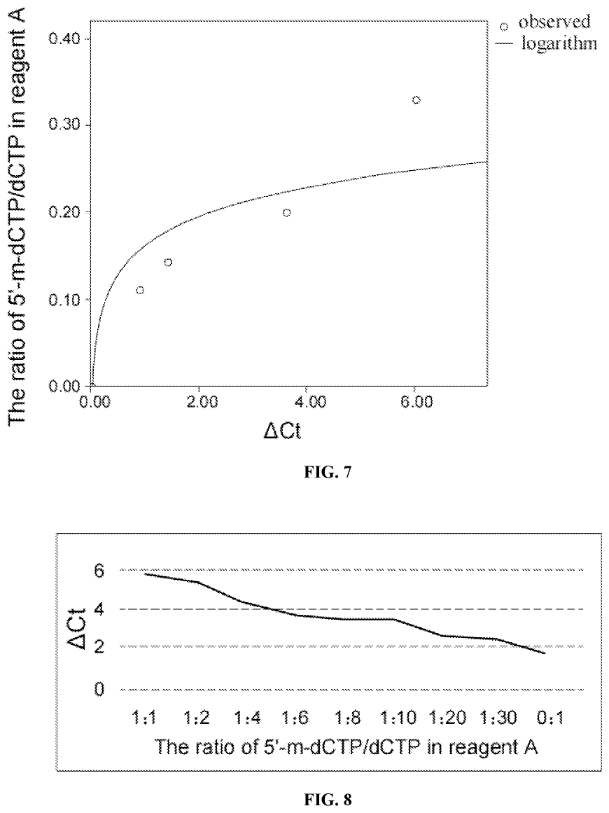

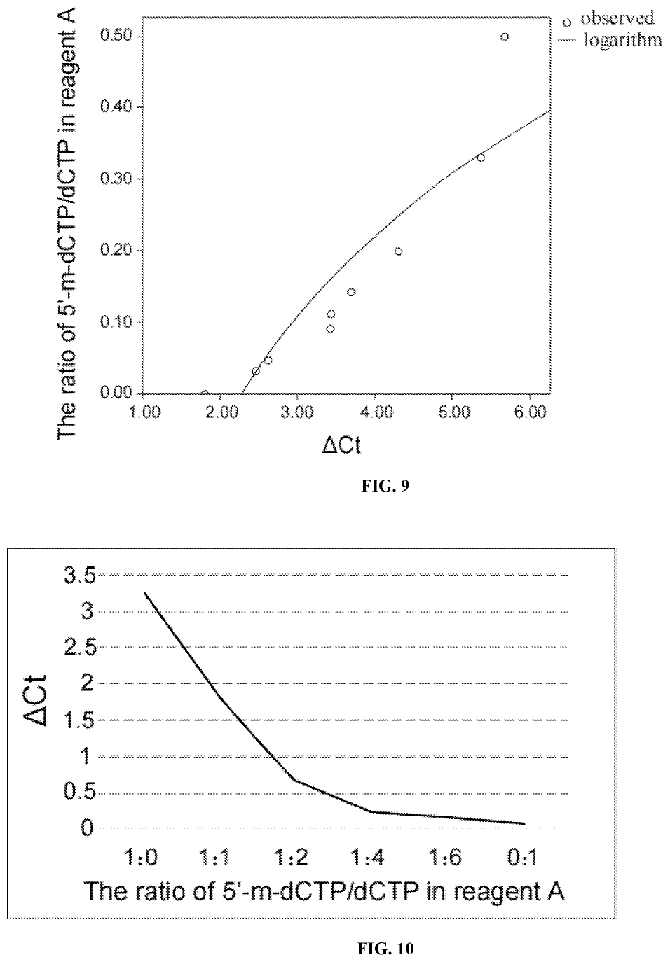

According to the above methylation detection method, different DNA fragments with different methylation levels were tested further, and ΔCt values were compared. Results were shown in Tables 5 to Table 8 and FIG. 2 to FIG. 9.

TABLE 5
Detection of SHOX2 gene fragments
with different methylation levels

| | Methylated DNA fragment (reagent A: 5′-m-dCTP:dCTP) | | | | | | |
|---|---|---|---|---|---|---|---|
| | 1:1 | 1:2 | 1:4 | 1:6 | 1:8 | 1:10 | 0:1 |
| Ct2 | 20.31 | 17.36 | 15.79 | 15.47 | 15.59 | 14.12 | 14.16 |
| Ct1 | 15.09 | 14.9 | 14.73 | 14.99 | 15.08 | 14.06 | 14.12 |
| ΔCt | 5.22 | 2.46 | 1.06 | 0.48 | 0.51 | 0.06 | 0.04 |

TABLE 6
Detection of P53 gene fragments with different methylation levels

| | Methylated DNA fragment (reagent A: 5′-m-dCTP:dCTP) | | | | | | | | | |
|---|---|---|---|---|---|---|---|---|---|---|
| | 1:0 | 1:1 | 1:2 | 1:4 | 1:6 | 1:8 | 1:10 | 1:20 | 1:30 | 0:1 |
| Ct2 | 24.43 | 22.28 | 21.17 | 19.89 | 19.47 | 18.51 | 18.09 | 17.00 | 16.94 | 16.71 |
| Ct1 | 18.75 | 16.74 | 16.33 | 16.33 | 16.10 | 16.14 | 15.91 | 15.29 | 15.70 | 15.35 |
| ΔCt | 5.68 | 5.54 | 4.84 | 3.56 | 3.37 | 2.37 | 2.18 | 1.71 | 1.24 | 1.36 |

TABLE 7
Detection of APC gene fragments with different methylation levels

| | Methylated DNA fragment (reagent A: 5′-m-dCTP:dCTP) | | | | |
|---|---|---|---|---|---|
| | 1:2 | 1:4 | 1:6 | 1:8 | 0:1 |
| Ct2 | 21.87 | 19.73 | 17.33 | 16.7 | 16.2 |
| Ct1 | 15.83 | 16.09 | 15.88 | 15.78 | 16.18 |
| ΔCt | 6.04 | 3.64 | 1.45 | 0.92 | 0.02 |

TABLE 8
Detection of Apobec3B gene fragments
with different methylation levels

| | Methylated DNA fragment (reagent A: 5′-m-dCTP:dCTP) | | | | | | | | |
|---|---|---|---|---|---|---|---|---|---|
| | 1:1 | 1:2 | 1:4 | 1:6 | 1:8 | 1:10 | 1:20 | 1:30 | 0:1 |
| Ct2 | 26.03 | 24.34 | 22.72 | 21.76 | 22.62 | 20.71 | 19.73 | 19.68 | 18.85 |
| Ct1 | 20.35 | 18.97 | 18.41 | 18.06 | 19.18 | 17.28 | 17.10 | 17.21 | 17.04 |
| ΔCt | 5.68 | 5.37 | 4.31 | 3.7 | 3.44 | 3.43 | 2.63 | 2.47 | 1.81 |

The above results showed that the ΔCt in four different methylated DNA fragments is in proportion to the methylation level even though different genes exhibit different limit values, and it was inferred that the detection method is suitable for the detection of all methylated DNA fragments. DNA fragments with different methylation levels each were compared with a corresponding non-methylated DNA fragment (regent A, 5′-m-dCTP : dCTP=0: 1), and comparison results showed that different methylated gene fragments corresponded to different detection limit values.

### Example 2

Method for Detecting cf/ctDNA Methylation in Lung Cancer
1) Identification of Target Genes In Lung Cancer:

Bioinformatics analysis: According to the analysis of the Cancer Genome Atlas (TCGA) database, HOXD12 was identified to display a large difference for methylation between healthy lung tissue and lung cancer tissue; furthermore, the specificity was evaluated by a random forest statistical analysis model, and indicated that this gene contributed the most to the identification of lung cancer. HOXD12 is a member of the homologous box gene family, which plays an important regulatory role in morphogenesis. Current studies have shown that different members of the homologous box gene family all played an important role in the occurrence and development of a tumor.

Given that the culprit of tumorigenesis is bonded to gene instability, so gene analysis related to genomic instability was conducted: Studies have shown that cytosine deaminase (CD) could convert cytosine into uracil through the deamination of cytosine, and the abnormal expression of this gene could lead to various mutations of DNA, which was an important factor causing genomic instability and tumorigenesis. Studies have also shown that AID underwent abnormal high expression in a variety of tumors, especially in lung cancer; many experiments have shown that the overexpression of this gene could significantly increase the genomic mutation frequency; and transgenic animal experiments have shown that the overexpression of AID could cause time-dependent dysplasia and thus lead to a tumor. There have been currently a lot of reports on the relationship between this gene and lung cancer, which mainly focus on the study of an expression difference of the gene; and the study of a methylation difference of the gene has been rarely reported.

The quantity of cell-free DNA (cfDNA) in plasma: It is known that a metabolism difference between tumor patient and healthy individual, which elicits a difference of DNA sum in peripheral blood between healthy individual and tumor patient. So, the quantity of cell-free DNA in plasma is another marker of tumor progressing, the surrogate for evaluating sum of cfDNA in plasma was investigated. Human genomic analysis shown that Alu gene, a highly repetitive sequence in genome, is the highest copy number in the human genome, so this gene can act as an ideal surrogate for genome quantization.
2) Primer and probe design for target regions of different target genes
(1) Primer and probe design: The software Primer 5 was used to design promoter regions of AID and HOXD12 genes, in which the other parameters such as number and distribution of CG islands and length of an amplified fragment were also optimized. For Alu fragment: by alignment analysis, a conserved region sequence in the Alu genosome was chosen and designed by Primer 5. Primer and probe synthesis: these sequences were synthesized by Sangon Biotech (Shanghai) Co., Ltd.
(2) Experimental samples: Healthy samples (plasma) were collected from individuals undergoing physical examination in Beijing Jiaotong University Hospital; and lung cancer samples (plasma) were collected from the Cancer Center Laboratory of the First Medical Center of the PLA General Hospital. 42 healthy samples and 53 lung cancer samples were randomly selected. The extraction of plasma cfDNA was completed using a cfDNA detection kit of Magen Biotechnology Co., Ltd.
(3) Experimental verification: The candidate primer pair and probes sequences were confirmed for their sensitivity and specificity by PCR with the templates of cfDNA extracted from plasma. The condition of PCR parameters were optimized further through experiments to screen ideal primers and probes . The qPCR was implemented in Hongshi SLAN 96P instrument and PCR conditions were as follows: (1) High-temperature denaturation (HT): 3 gene fragments (AID/HOXD12/Alu): predenaturation at 95° C. for 5 min; and then three-step amplification: 94° C. for 5 s, 60° C. for 15 s, and 72° C. for 30 s, with 45 cycles. (2) Low-temperature denaturation (LT): 2 gene fragments (AID/HOXD12): 85° C. for 15 s, 60° C. for 15 s, and 72° C. for 30 s, with 18 cycles; and then the following cyclic program: 94° C. for 5 s, 60° C. for 15 s, and 72° C. for 30 s, with 27 cycles.

TABLE 9
Primer and probe sequences for target genes detection by qPCR

| | Upstream primer (5′-3′) | Downstream primer (5′-3′) | Probe (5′FAM-3′BHQ1) |
|---|---|---|---|
| AID | ttgaagtgtctactgttactgcc | ctgtcataggcagagtcacaca | ctagctatggagcatggactgggc |
| | (SEQ ID NO: 13) | (SEQ ID NO: 14) | (SEQ ID NO: 15) |
| HOXD12 | cagcctcgtccttcgccat | ataagaggacaggctcagacgat | cagcaccgcctacatccccccgacccg |
| | (SEQ ID NO: 16) | (SEQ ID NO: 17) | (SEQ ID NO: 18) |
| Alu | gtggctcacgcctgtaatc | ttagtagagacggggtttca | ttgggaggccgaggcgggcggat |
| | (SEQ ID NO: 19) | (SEQ ID NO: 20) | (SEQ ID NO: 21) |

Notes:
Lengths of PCR-amplified fragments of target regions of target genes:
AID (promoter region): 145 bp (5′-ttgaagtgtctactgttactgcctcctgatctttgctagctatggagcatggactgggctttta
gagcagcagccccaaaggaacctaaacattaaagcagagctgccctcaatggtttaacctgtgtgactctgcctatgacag-3′,
SEQ ID NO: 35);
HOXD12 (promoter region): 155 bp (5′-cagcctcgtccttcgccatcgaacattgcgggtgttatcataatactctgaagggggggaa
aacgggtcggggggatgtaggcggtgctgaaatgaccggctttgaagaacctgcaggcaaagtttcgtccaatcgtctgagcctgtcctcttat-3′,
SEQ ID NO: 36);
Alu (genosome): 112 bp (5′-gtggctcacgcctgtaatcccagcactttgggaggccgagggggcggatcacctgaggtcaggagt
tcgagacc agcctggccaacatggtgaaaccccgtctctactaaa-3′, SEQ ID NO: 37).

3) Analysis of a Dose-Response Relationship between Detection Values and Methylation of Amplified Target Regions in Different Target Genes:

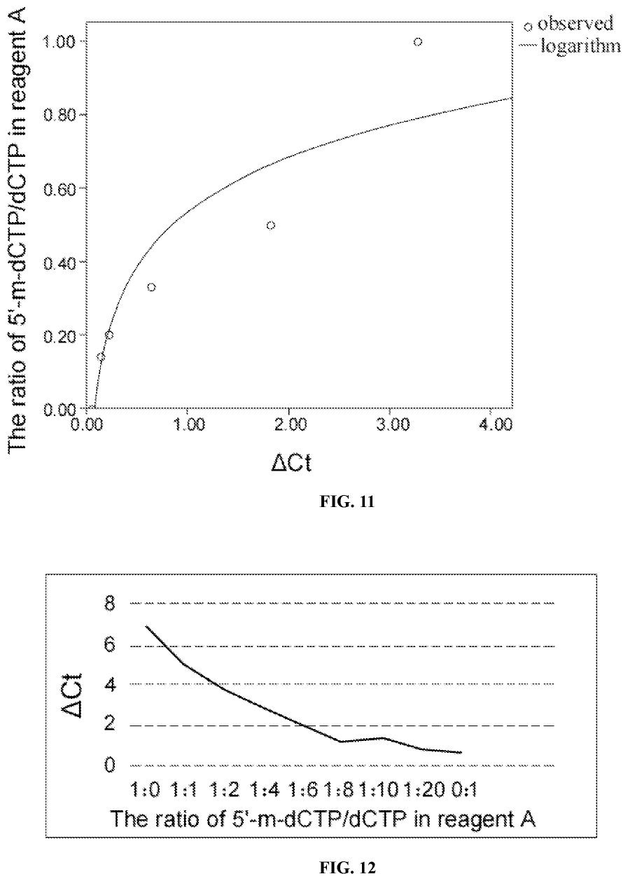

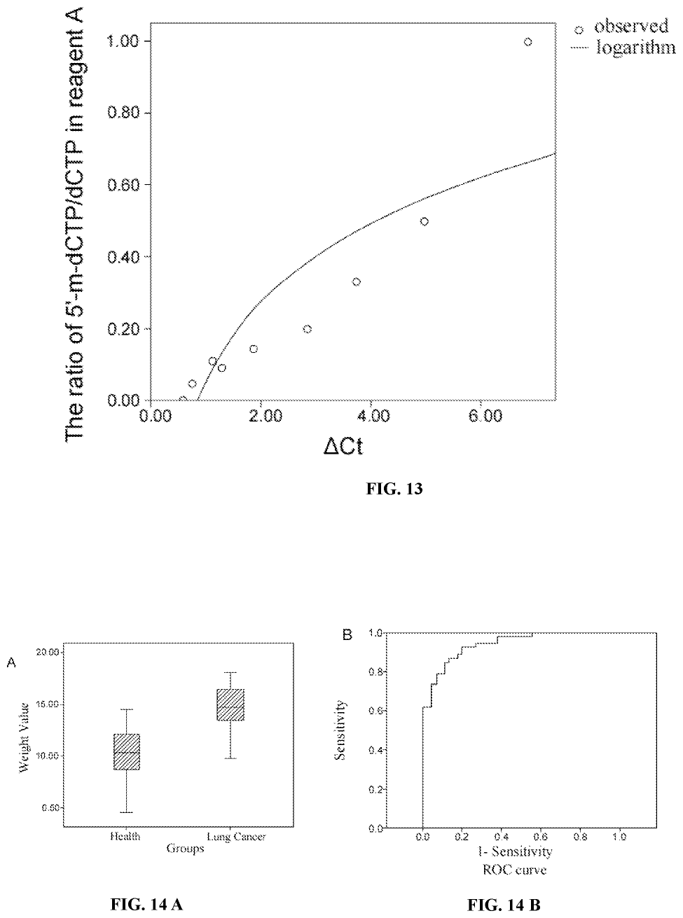

In order to further verify the dose-response relationship between methylation and detection values of different target regions, a methylation reference curve was first established for different target regions, and test results of different methylation levels of different genes were shown in Table 10 and 11 and FIG. 10 to FIG. 13.

TABLE 10
Detection of different methylation levels of the AID gene by qPCR

| | Methylated DNA fragment (reagent A: 5′-m-dCTP:dCTP) | | | | | |
|---|---|---|---|---|---|---|
| | 1:0 | 1:1 | 1:2 | 1:4 | 1:6 | 0:1 |
| Ct2 | 22.54 | 22.71 | 20.16 | 20.94 | 23.26 | 23.11 |
| Ct1 | 19.26 | 20.88 | 19.51 | 20.71 | 23.11 | 23.05 |
| ΔCt | 3.28 | 1.83 | 0.65 | 0.23 | 0.15 | 0.06 |

TABLE 11
Detection of different methylation
levels of the HOXD12 gene by qPCR

| | Methylated DNA fragment (reagent A: 5′-m-dCTP:dCTP) | | | | | | | | |
|---|---|---|---|---|---|---|---|---|---|
| | 1:0 | 1:1 | 1:2 | 1:4 | 1:6 | 1:8 | 1:10 | 1:20 | 0:1 |
| Ct2 | 22.87 | 25.1 | 20.58 | 20.05 | 19.21 | 18.67 | 18.1 | 16.34 | 17.1 |
| Ct1 | 16 | 20.12 | 16.84 | 17.2 | 17.34 | 17.54 | 16.8 | 15.58 | 16.51 |
| ΔCt | 6.87 | 4.98 | 3.74 | 2.85 | 1.87 | 1.13 | 1.3 | 0.76 | 0.59 |

4) Identification of Alu as a surrogate of genomic abundance: When Alu was subjected to a gene abundance test, it was found that a Ct value of either a healthy individual or a lung cancer patient did not exceed 20, and thus in order to keep the unit consistent, 20 was subtracted by a Ct value of Alu to obtain a corresponding ΔCt value: ΔCt=20−Ct.
5) Combined analysis and weight value analysis of multiple target genes

Ct values obtained under two conditions for each of the AID and HOXD12 target genes were subtracted to obtain a ΔCt value (Ct2-Ct1) respectively. The ΔCt value of Alu was a result of subtracting 20 by a tested Ct value of Alu. ΔCt values of three genes in the healthy individual and lung cancer patient were subjected to statistical analysis to obtain a weight value equation and threshold for joint detection of the three genes (AID, HOXD12, and Alu), and the sensitivity and specificity of the detection were analyzed by an ROC curve. A weight value of a sample was calculated according to the following equation:

weight value=1.325×ΔCt_{AID}+2.084×ΔCt_{HOXD12}+2.152×ΔCt_{Alu}; and

the threshold was 12.468.

Test data of the healthy and lung cancer samples were shown in table 20 (data in gray font in the table indicated false positives) and Table 21 (data in gray font in the table indicated false negatives). The test data of the healthy and lung cancer samples were compared with the threshold to obtain threshold-based evaluation results of the lung cancer and healthy samples (as shown in Table 12). Evaluation results were subjected to T-test analysis, and analysis results were shown in Table 13.

TABLE 12
Threshold-based evaluation results of
the lung cancer and healthy samples

| | | Test group | | | |
|---|---|---|---|---|---|
| | | | Lung | | |
| | | Healthy | cancer | Total | Percentage (%) |
| Pathological | Healthy | 39 | 5 | 44 | 88.6 |
| group | Lung | 6 | 45 | 51 | 88.2 |
| | cancer | | | | |
| Total | | 45 | 50 | 95 | 88.4 |

6) Threshold-based analysis of the significance of a difference between samples of the two groups:

TABLE 13
T-test analysis between lung cancer and healthy samples
Group statistics

| | | Number | Mean | Standard | Standard error of |
|---|---|---|---|---|---|
| | Group | of cases | value | deviation (SD) | mean (SEM) |
| Weight | Healthy | 44 | 10.1804 | 2.36949 | 0.35322 |
| value | Lung | 51 | 14.8269 | 1.91125 | 0.26253 |
| | cancer | | | | |

Statistical results showed that a mean value of weight values in the healthy samples was significantly different from a mean value of weight values in the lung cancer samples (P value=0.000), indicating that there was a significant difference in DNA methylation of target genes between the lung cancer samples and the healthy samples.

Sensitivity analysis: Among the 51 samples of lung cancer patients, 6 had false negatives, sensitivity=(51-6)/51=88.23%.

Specificity analysis: Among the 45 healthy individuals, 5 had false positives, specificity=(45-5)/44=88.6%.

Coincidence rate=[(51−6)+(45−5)]/(51+44)=88.4%.

The sensitivity and specificity were further subjected to Receiver Operating Characteristic analysis (ROC curve), and an area under the curve (AUC) 0.96 indicates that the detection technique has high sensitivity and specificity. The distribution of weight values in the healthy and lung cancer samples and the ROC curve for the sensitivity and specificity analysis were shown in FIG. 14A and FIG. 14B respectively.

### Example 3

Detection of cf/ctDNA Methylation in CRC
1) Identification of target genes for CRC:

Bioinformatics analysis: According to the analysis of a TCGA database, the candidate genes with difference in the methylation between healthy tissue and CRC tissue was screened; Candidate genes were analyzed by a random forest statistical model, three genes SDC2/WDR17/ADHFE1 contributed greatly to the identification of CRC were selected. The methylation of promoter regions of the three genes was then tested according to the above method.

The quantity of cell-free DNA (cfDNA) in plasma: It is known that a metabolism difference between tumor patient and healthy individual, which elicits a difference of DNA sum in peripheral blood between healthy individual and tumor patient. So, the quantity of cell-free DNA in plasma is another marker of tumor progressing, the surrogate for evaluating sum of cfDNA in plasma was investigated. Human genomic analysis shown that Alu gene, a highly repetitive sequence in genome, is the highest copy number in the human genome, so this gene can act as an ideal surrogate for genome quantization.
2) Primer and probe design for target regions of different target genes:
(1) Primer and probe design: The software Primer 5 was used to design promoter regions of the SDC2/WDR17/ADHFE1 genes, in which the other parameters such as number and distribution of CG islands and length of an amplified fragment were also optimized. For Alu fragment: by aligning analysis, a conserved region sequence in the Alu gene was chosen and designed by Primer 5. Primers and probes were synthesized by Sangon Biotech (Shanghai) Co., Ltd.
(2) Experimental samples: Healthy samples (plasma) were collected from individuals undergoing physical examination in Beijing Jiaotong University Hospital; and CRC samples (plasma) were collected from the Cancer Center Laboratory of the First Medical Center of the PLA General Hospital. 54 healthy samples and 55 CRC samples were randomly selected. Extraction kit of plasma cfDNA was purchased from Magen Biotechnology Co., Ltd.
(3) Experimental verification: The specificity and sensitivity of candidate primer pairs and probes were verified by PCR and condition was optimized to screen ideal primer pairs and probes. qPCR was implemented in Hongshi SLAN 96P instrument and PCR conditions were as follows: (1) High-temperature denaturation (HT): four gene fragments (SDC2/WDR17/ADHFE1/Alu): predenaturation at 95° C. for 5 min; and then three-step amplification: 94° C. for 5 s, 62° C. for 15 s, and 72° C. for 30s, with 45 cycles. (2) Low-temperature denaturation (LT): WDR17/ADHFE1 gene fragments: 86° C. for 15 s, 62° C. for 15 s, and 72° C. for 30 s, with 16 cycles; and then the following cyclic program: 94° C. for 5 s, 60° C. for 15 s, and 72° C. for 30 s, with 29 cycles. SDC2 gene fragment: 88° C. for 15 s, 62° C. for 15 s, and 72° C. for 30 s, with 16 cycles; and then the following cyclic program: 94° C. for 5 s, 60° C. for 15 s, and 72° C. for 30 s, with 29 cycles.

TABLE 14
Primer and probe sequences for target genes detection by qPCR

| | Upstream primer (5′-3′) | Downstream primer (5′-3′) | Probe (5′FAM-3′BHQ1) |
|---|---|---|---|
| WDR17 | agggtcacgagccacttcc | ggagccacgatggaaggc | cctctcgggcgctccaatcagcg |
| | (SEQ ID NO: 22) | (SEQ ID NO: 23) | (SEQ ID NO: 24) |
| SDC2 | tcggcgtgtaatcctgtagg | ccctcctgagcctgcttc | cgggctcccctgggcgactggg |
| | (SEQ ID NO: 25) | (SEQ ID NO: 26) | (SEQ ID NO: 27) |
| ADHFE1 | agatttaaggcagaacccgag | gtcacctgtctaagcctcaag | cagtcgggctcagggccattttccc |
| | (SEQ ID NO: 28) | (SEQ ID NO: 29) | (SEQ ID NO: 30) |
| Alu | gtggctcacgcctgtaatc | ttagtagagacggggtttca | ttgggaggccgaggcgggggat |
| | (SEQ ID NO: 19) | (SEQ ID NO: 20) | (SEQ ID NO: 21) |

Notes:
Lengths of PCR-amplified fragments of target regions in target genes:
WDR17 (promoter region): 124 bp: (5′-agggtcacgagccacttccgcctccccctctcgggcgctccaatcagcgacctgtgta
gctgtcacgtgggcctctggggcagaccctggagatcgggctcgcggcgccttccatcgtggctcc-3′, SEQ ID NO: 38);
SDC2 (promoter region): 107 bp: (5′-tcggcgtgtaatcctgtaggaatttctcccgggtttatctgggagtcacactgccgcct
cctctccccagtcgcccaggggagcccggagaagcaggctcaggaggg-3′, SEQ ID NO: 39);
ADHFE1 (promoter region): 157 bp: (5′-
agatttaaggcagaacccgaggcctacggagcagttaccttctacggcaatttcaagggtggatggtgcgagcgccgctggggcagctggcgttc
tggttcttactccgtgggaaaatggccctgagcccgactggcttgaggcttagacaggtgac-3′, SEQ ID NO: 40); and
Alu (genosome): 112 bp: (5′-gtggctcacgcctgtaatcccagcactttgggaggccgaggcgggcggatcacctgaggtcaggagt
tcgagaccagcctggccaacatggtgaaaccccgtctctactaaa-3′, SEQ ID NO: 37).

3) Analysis of a dose-response relationship between detection values and methylation of amplified target regions in different target genes:

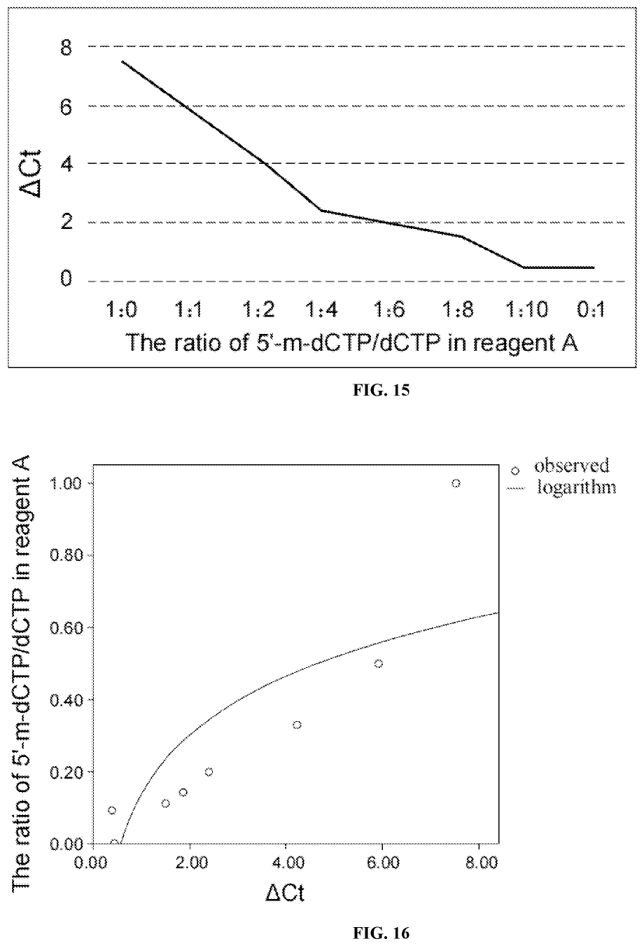

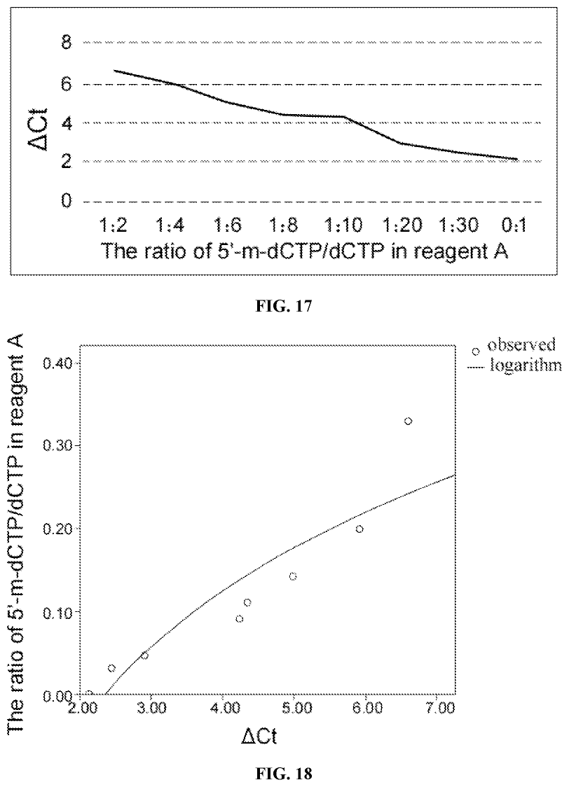

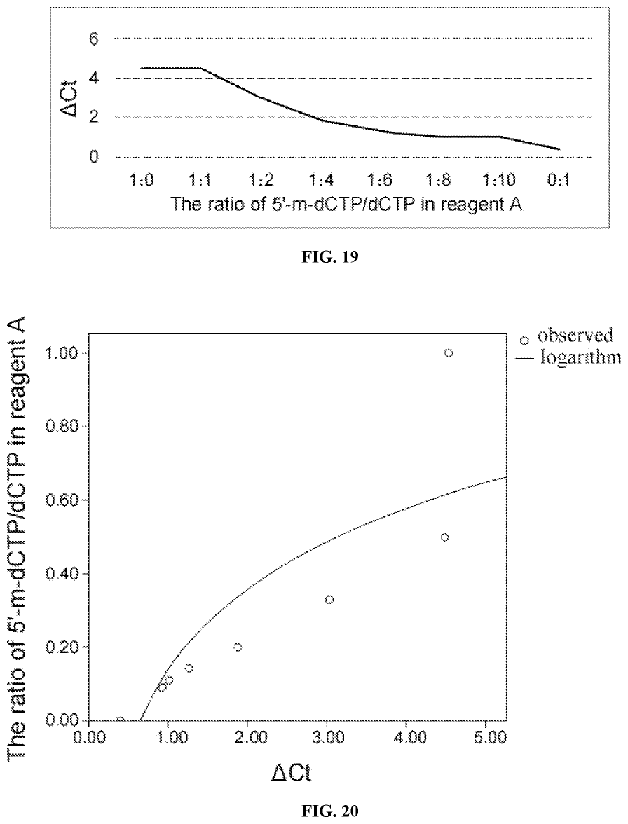

In order to further verify the dose-response relationship between methylation and detection values of different target regions, a methylation reference curve was first established for different target regions, and test results of different methylation levels of different genes were shown in Table 15 to Table 17 and FIG. 15 to FIG. 20.

TABLE 15
Test data of different methylation levels of the SDC2 gene

| | Methylated DNA fragment (reagent A: 5′-m-dCTP:dCTP) | | | | | | | |
|---|---|---|---|---|---|---|---|---|
| | 1:0 | 1:1 | 1:2 | 1:4 | 1:6 | 1:8 | 1:10 | 0:1 |
| Ct2 | 21.72 | 17.39 | 16.25 | 13.55 | 13.11 | 13.36 | 12.05 | 11.75 |
| Ct1 | 14.19 | 11.47 | 12.02 | 11.15 | 11.25 | 11.86 | 11.65 | 11.31 |
| ΔCt | 7.53 | 5.92 | 4.23 | 2.40 | 1.86 | 1.50 | 0.40 | 0.44 |

TABLE 16
Test data of different methylation levels of the WDR17 gene

| | Methylated DNA fragment (reagent A: 5′-m-dCTP:dCTP) | | | | | | | |
|---|---|---|---|---|---|---|---|---|
| | 1:2 | 1:4 | 1:6 | 1:8 | 1:10 | 1:20 | 1:30 | 0:1 |
| Ct2 | 30.05 | 25.68 | 25.25 | 25.21 | 26.92 | 19.66 | 19.38 | 22.32 |
| Ct1 | 23.45 | 19.76 | 20.26 | 20.86 | 22.68 | 16.75 | 16.93 | 20.19 |
| ΔCt | 6.6 | 5.92 | 4.99 | 4.35 | 4.24 | 2.91 | 2.45 | 2.13 |

TABLE 17
Test data of different methylation levels of the ADHFE1 gene

| | Methylated DNA fragment (reagent A: 5′-m-dCTP:dCTP) | | | | | | | |
|---|---|---|---|---|---|---|---|---|
| | 1:0 | 1:1 | 1:2 | 1:4 | 1:6 | 1:8 | 1:10 | 0:1 |
| Ct2 | 22.84 | 20.72 | 18.56 | 17.46 | 17.79 | 17.19 | 17.14 | 16.52 |
| Ct1 | 18.30 | 16.23 | 15.53 | 15.58 | 16.52 | 16.18 | 16.21 | 16.12 |
| ΔCt | 4.54 | 4.49 | 3.03 | 1.88 | 1.27 | 1.01 | 0.93 | 0.4 |

4) Identification of Alu as a surrogate of genomic abundance: When Alu was subjected to gene abundance test, it was found that the Ct value of either healthy individual or CRC patient did not exceed 20, and thus in order to keep the unit consistent, 20 was subtracted by a Ct value of Alu to obtain a corresponding ΔCt value: ΔCt=20−Ct.
5) Determination of a difference between two populations (combined analysis and weight value analysis of target genes)

Ct values obtained under two conditions for each target gene were subtracted to obtain a ΔCt value (Ct2−Ct1) respectively. A ΔCt value of Alu was a result of subtracting 20 by a tested Ct value of Alu. ΔCt values of four genes in the healthy individual and CRC patient were subjected to statistical analysis to obtain a weight value equation and threshold for joint detection of the four genes, and the sensitivity and specificity of the detection were analyzed by an ROC curve. A weight value of a sample was calculated according to the following equation III:

weight value=3.35×ΔCt_{SDC2}+0.99×ΔCt_{WDR17}+0.933×ΔCt_{ADHEF1}+1.677×ΔCt_{Alu} equation III; and

the threshold was 12.173.

The test data of the healthy and CRC samples were compared with the threshold to obtain evaluation results. Test results of CRC patients were shown in Table 22 (data shaded in grayscale indicated false negatives); and test results of healthy individuals were shown in Table 23 (data shaded in grayscale indicated false positives). A statistics analysis of result in two groups was shown in Table 18. Evaluation results in two groups were subjected to T-test analysis, and results were shown in Table 19.

TABLE 18
Threshold-based evaluation results of the
two groups (CRC and healthy samples)

| | | Test group | | | |
|---|---|---|---|---|---|
| | | Healthy | CRC | Total | Percentage (%) |
| Pathological | Healthy | 51 | 3 | 54 | 94.4 |
| group | CRC | 8 | 49 | 57 | 85.9 |

| | | | | |
|---|---|---|---|---|
| Total | 59 | 52 | 111 | 90.1 |

Sensitivity = 85.9%, specificity = 94.4%, and compliance rate = 90.1%.

TABLE 19
T-test analysis of evaluation results

| Group | Number of cases | Mean value | SD | SEM |
|---|---|---|---|---|
| CRC | 57 | 16.6525 | 5.20192 | 0.68901 |
| Healthy | 54 | 9.3080 | 2.35822 | 0.31513 |

Statistical results showed that a mean value of weight values of the healthy samples was significantly different from a mean value of weight values of the CRC samples (P value=0.000), indicating that there was a significant difference in DNA methylation of target genes between the CRC samples and the healthy samples.

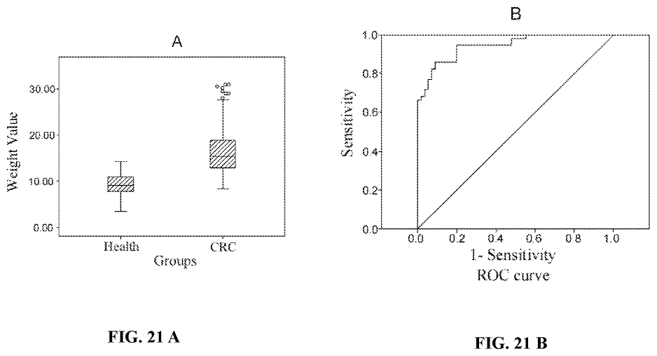

The mean values in two groups were plotted as FIG. 21A. The sensitivity and specificity were evaluated by Receiver Operating Characteristic Analysis (ROC) with an AUC 0.97 (FIG. 21B), indicating that the detection technique has high sensitivity and specificity.

The above description of examples is merely provided to help illustrate the method of the present disclosure and a core idea thereof. It should be noted that several improvements and modifications may be made by persons of ordinary skill in the art without departing from the principle of the present disclosure, and these improvements and modifications should also fall within the protection scope of the present disclosure. Various modifications to these examples are apparent to those of professional skill in the art, and the general principles defined herein may be implemented in other examples without departing from the spirit or scope of the present disclosure. Thus, the present disclosure is not limited to the examples shown herein but falls within the widest scope consistent with the principles and novel features disclosed herein.

TABLE 20
Test data of healthy samples for paired with lung cancer group

| | | | | | | | A LU | | | |
|---|---|---|---|---|---|---|---|---|---|---|
| Healthy | A ID | | | HO XD 12 | | | Fixed | | | Weight |
| sample | Ct2 | Ct1 | ΔCt | Ct2 | Ct1 | ΔCt | value | Ct1 | ΔCt | value |
| N1 | 33.64 | 32.52 | 1.12 | 37.22 | 35.8 | 1.42 | 20 | 19.78 | 0.22 | 4.91672 |
| N2 | 33.99 | 32.61 | 1.38 | 35.88 | 34.89 | 0.99 | 20 | 17.42 | 2.58 | 9.44382 |
| N3 | 32.99 | 32.17 | 0.82 | 35.39 | 34.16 | 1.23 | 20 | 17.82 | 2.18 | 8.34118 |
| N4 | 33.31 | 32.45 | 0.86 | 35.55 | 33.92 | 1.63 | 20 | 18.5 | 1.5 | 7.76442 |
| N5 | 33.32 | 33.09 | 0.23 | 34.09 | 33.95 | 0.14 | 20 | 16.44 | 3.56 | 8.25763 |
| N6 | 33.2 | 32.64 | 0.56 | 35.32 | 34.43 | 0.89 | 20 | 17.71 | 2.29 | 7.52484 |
| N7 | 33.43 | 32.69 | 0.74 | 35.78 | 34.85 | 0.93 | 20 | 17.42 | 2.58 | 8.47078 |
| N8 | 32.45 | 31.55 | 0.9 | 35.75 | 33.84 | 1.91 | 20 | 16.02 | 3.98 | [symbol] |
| N9 | 33.65 | 31.88 | 1.77 | 34.08 | 33.14 | 0.94 | 20 | 18.32 | 1.68 | 7.91957 |
| N10 | 33.17 | 31.07 | 2.1 | 35.18 | 34.04 | 1.14 | 20 | 17.42 | 2.58 | 10.71042 |
| N11 | 32.68 | 31.08 | 1.6 | 35.62 | 34.09 | 1.53 | 20 | 17.29 | 2.71 | 11.14044 |
| N12 | 31.21 | 30.89 | 0.32 | 36.11 | 34.25 | 1.86 | 20 | 17.73 | 2.27 | 9.18528 |
| N13 | 34.07 | 32.55 | 1.52 | 36.26 | 34.82 | 1.44 | 20 | 17.52 | 2.48 | 10.35192 |
| N14 | 32.98 | 32.37 | 0.61 | 35.15 | 33.36 | 1.79 | 20 | 17.12 | 2.88 | 10.73637 |
| N15 | 34.61 | 33.63 | 0.98 | 36.53 | 34.86 | 1.67 | 20 | 18.09 | 1.91 | 8.8891 |
| N16 | 32.96 | 32.31 | 0.65 | 34.04 | 33.12 | 0.92 | 20 | 15.83 | 4.17 | 11.75237 |
| N17 | 31.98 | 32 | −0.02 | 35.87 | 35 | 0.87 | 20 | 17.27 | 2.73 | 7.66154 |
| N18 | 34.75 | 33.48 | 1.27 | 37.12 | 34.47 | 2.65 | 20 | 17.16 | 2.84 | [symbol] |
| N19 | 33.7 | 33.1 | 0.6 | 35.85 | 34.09 | 1.76 | 20 | 16.3 | 3.7 | 12.42524 |
| N20 | 31.7 | 30.02 | 1.68 | 35.72 | 33.94 | 1.78 | 20 | 17.14 | 2.86 | 12.09024 |
| N21 | 32.13 | 31.62 | 0.51 | 32.73 | 31.7 | 1.03 | 20 | 15.06 | 4.94 | [symbol] |
| N22 | 34.04 | 33.21 | 0.83 | 35.06 | 34.03 | 1.03 | 20 | 17.33 | 2.67 | 8.99211 |
| N23 | 32.44 | 32.03 | 0.41 | 33.48 | 32.16 | 1.32 | 20 | 16.51 | 3.49 | 10.80461 |
| N24 | 33.7 | 33.66 | 0.04 | 35.43 | 33.65 | 1.78 | 20 | 17.51 | 2.49 | 9.121 |
| N25 | 32.78 | 32.45 | 0.33 | 34.88 | 32.73 | 2.15 | 20 | 16.63 | 3.37 | 12.17009 |
| N26 | 33.6 | 33.8 | −0.2 | 35.78 | 33.85 | 1.93 | 20 | 17.01 | 2.99 | 10.1916 |
| N27 | 33.34 | 33.25 | 0.09 | 35.3 | 34.4 | 0.9 | 20 | 18.27 | 1.73 | 5.71781 |
| N28 | 36.68 | 33.28 | 3.4 | 36.25 | 34.23 | 2.02 | 20 | 17.32 | 2.68 | [symbol] |
| N29 | 35.2 | 33.86 | 1.34 | 34.76 | 34.15 | 0.61 | 20 | 16.55 | 3.45 | 10.47114 |
| N30 | 33.32 | 32.51 | 0.81 | 34.84 | 34.04 | 0.8 | 20 | 17.08 | 2.92 | 9.02429 |
| N31 | 33.04 | 32.88 | 0.16 | 35.87 | 34.31 | 1.56 | 20 | 17.57 | 2.43 | 8.6924 |
| N32 | 33.53 | 32.29 | 1.24 | 35.55 | 33.92 | 1.63 | 20 | 16.39 | 3.61 | [symbol] |
| N33 | 31.67 | 29.75 | 1.92 | 35.5 | 33.79 | 1.71 | 20 | 16.12 | 3.88 | 14.4574 |
| N34 | 34.59 | 33.06 | 1.53 | 35.2 | 33.87 | 1.33 | 20 | 16.55 | 3.45 | 12.22337 |
| N35 | 33.31 | 31.5 | 1.81 | 35.03 | 33.42 | 1.61 | 20 | 17.77 | 2.23 | 10.55245 |
| N36 | 32.88 | 30.32 | 2.56 | 37.06 | 35.07 | 1.99 | 20 | 17.58 | 2.42 | 12.747 |
| N37 | 34.13 | 33.27 | 0.86 | 35.12 | 33.62 | 1.5 | 20 | 16.66 | 3.34 | 11.45318 |
| N38 | 33.12 | 32.35 | 0.77 | 35.59 | 35.29 | 0.3 | 20 | 18.64 | 1.36 | 4.57217 |
| N39 | 32.93 | 32.39 | 0.54 | 35.34 | 33.9 | 1.44 | 20 | 17.17 | 2.83 | 9.80662 |
| N40 | 33.93 | 33.27 | 0.66 | 36.1 | 34.71 | 1.39 | 20 | 17.4 | 2.6 | 9.36646 |
| N41 | 31.07 | 30.33 | 0.74 | 34.09 | 32.34 | 1.75 | 20 | 16.76 | 3.24 | 11.59998 |
| N42 | 33.64 | 34.12 | −0.48 | 34.49 | 33.05 | 1.44 | 20 | 16.63 | 3.37 | 9.6172 |
| N43 | 34.32 | 34.23 | 0.09 | 35.62 | 34.69 | 0.93 | 20 | 17.49 | 2.51 | 7.45889 |
| N44 | 34.03 | 34.15 | −0.12 | 35.9 | 33.89 | 2.01 | 20 | 16.79 | 3.21 | 10.93776 |

TABLE 21
Test data of lung cancer samples

| | | | | | | | A LU | | | |
|---|---|---|---|---|---|---|---|---|---|---|
| Lung cancer | A ID | | | HO XD12 | | | Fixed | | | Weight |
| sample | Ct2 | Ct1 | ΔCt | Ct2 | Ct1 | ΔCt | value | Ct1 | ΔCt | value |
| T1 | 32.7 | 31.37 | 1.33 | 33.85 | 32.38 | 1.47 | 20 | 15.51 | 4.49 | 14.48821 |
| T2 | 32.44 | 31.12 | 1.32 | 33.91 | 32.49 | 1.42 | 20 | 15.44 | 4.56 | 14.5214 |
| T3 | 32.86 | 30.73 | 2.13 | 35.1 | 33.09 | 2.01 | 20 | 15.84 | 4.16 | 15.96341 |
| T4 | 32.16 | 31.18 | 0.98 | 33.42 | 31.12 | 2.3 | 20 | 16.22 | 3.78 | 14.22626 |
| T5 | 32.18 | 30.69 | 1.49 | 34.04 | 32.16 | 1.88 | 20 | 14.99 | 5.01 | 16.67369 |
| T6 | 33.56 | 30.6 | 2.96 | 37.46 | 34.85 | 2.61 | 20 | 16.77 | 3.23 | 16.3122 |
| T7 | 33.33 | 32.13 | 1.2 | 37.06 | 33.72 | 3.34 | 20 | 16.42 | 3.58 | 16.25472 |
| T8 | 33.36 | 31.26 | 2.1 | 36.79 | 34.91 | 1.88 | 20 | 17.36 | 2.64 | [symbol] |
| T9 | 32.31 | 30.13 | 2.18 | 35.12 | 33.62 | 1.5 | 20 | 15.01 | 4.99 | 16.75298 |
| T10 | 32.39 | 30.43 | 1.96 | 34.45 | 33.08 | 1.37 | 20 | 16.45 | 3.55 | 13.09168 |
| T11 | 32.36 | 30.02 | 2.34 | 35.86 | 33.58 | 2.28 | 20 | 15.96 | 4.04 | 16.5461 |
| T12 | 32.17 | 29.71 | 2.46 | 35.35 | 34.04 | 1.31 | 20 | 16.23 | 3.77 | 14.10258 |
| T13 | 31.94 | 30.22 | 1.72 | 33.63 | 32.13 | 1.5 | 20 | 15.07 | 4.93 | 16.01436 |
| T14 | 32.34 | 30.79 | 1.55 | 34.82 | 33.04 | 1.78 | 20 | 16.23 | 3.77 | 13.87631 |
| T15 | 33.27 | 31.41 | 1.86 | 34.9 | 32.88 | 2.02 | 20 | 16.03 | 3.97 | 15.21762 |
| T16 | 34.52 | 31.08 | 3.44 | 35.15 | 33.04 | 2.11 | 20 | 16.28 | 3.72 | 16.96068 |
| T17 | 32.2 | 31.47 | 0.73 | 33.36 | 31.31 | 2.05 | 20 | 14.13 | 5.87 | 17.87169 |
| T18 | 34.25 | 32.59 | 1.66 | 36.95 | 34.69 | 2.26 | 20 | 17.09 | 2.91 | 13.17166 |
| T19 | 32 | 31.17 | 0.83 | 35.19 | 33.18 | 2.01 | 20 | 14.93 | 5.07 | 16.19923 |
| T20 | 32.91 | 31.21 | 1.7 | 34.96 | 33.06 | 1.9 | 20 | 15.11 | 4.89 | 16.73538 |
| T21 | 31.64 | 30.48 | 1.16 | 33.28 | 31.61 | 1.67 | 20 | 14.16 | 5.84 | 17.58496 |
| T24 | 34.2 | 32.29 | 1.91 | 35.18 | 32.75 | 2.43 | 20 | 16.69 | 3.31 | 14.71799 |
| T22 | 31.83 | 30.74 | 1.09 | 32.93 | 31.63 | 1.3 | 20 | 14.26 | 5.74 | 16.50593 |
| T23 | 32.62 | 32.27 | 0.35 | 33.24 | 31.9 | 1.34 | 20 | 15.79 | 4.21 | [symbol] |
| T24 | 33.79 | 32.26 | 1.53 | 36.8 | 33.82 | 2.98 | 20 | 15.94 | 4.06 | 16.97469 |
| T25 | 34.23 | 32.55 | 1.68 | 37.69 | 33.82 | 3.87 | 20 | 16.41 | 3.59 | 18.01676 |
| T26 | 32.65 | 31.22 | 1.43 | 35.18 | 32.75 | 2.43 | 20 | 16.1 | 3.9 | 15.35167 |
| T27 | 32.47 | 31.34 | 1.13 | 35.21 | 31.86 | 3.35 | 20 | 16.29 | 3.71 | 16.46257 |
| T28 | 33.17 | 31.32 | 1.85 | 34.89 | 32.76 | 2.13 | 20 | 16.26 | 3.74 | 14.93865 |
| T29 | 33.84 | 32.45 | 1.39 | 38.69 | 35.67 | 3.02 | 20 | 17.04 | 2.96 | 14.50535 |
| T30 | 33.31 | 32.22 | 1.09 | 34.86 | 32.76 | 2.1 | 20 | 16.6 | 3.4 | 13.13745 |
| T31 | 33.66 | 33.29 | 0.37 | 34.55 | 32.95 | 1.6 | 20 | 16.73 | 3.27 | [symbol] |
| T32 | 33.18 | 32.28 | 0.9 | 35.1 | 33.14 | 1.96 | 20 | 16.2 | 3.8 | 13.45474 |
| T33 | 33.24 | 31.99 | 1.25 | 35.25 | 33.29 | 1.96 | 20 | 16.12 | 3.88 | 14.09065 |
| T34 | 32.56 | 30.7 | 1.86 | 34.46 | 32.37 | 2.09 | 20 | 14.99 | 5.01 | 17.60158 |
| T35 | 33.16 | 32.14 | 1.02 | 35.86 | 33.12 | 2.74 | 20 | 16.16 | 3.84 | 15.32534 |
| T36 | 33.01 | 32.45 | 0.56 | 34.8 | 33.24 | 1.56 | 20 | 16.06 | 3.94 | 12.47192 |
| T37 | 33.18 | 31.53 | 1.65 | 36.29 | 33.52 | 2.77 | 20 | 15.49 | 4.51 | 17.66445 |
| T38 | 33.32 | 32.49 | 0.83 | 34.51 | 33.15 | 1.36 | 20 | 17.28 | 2.72 | [symbol] |
| T39 | 32.1 | 31.21 | 0.89 | 33.46 | 32.07 | 1.39 | 20 | 15.04 | 4.96 | 14.74993 |
| T40 | 30.92 | 29.7 | 1.22 | 36.2 | 34.04 | 2.16 | 20 | 16.57 | 3.43 | 13.4993 |
| T41 | 32.02 | 30.98 | 1.04 | 34.62 | 33.18 | 1.44 | 20 | 15.22 | 4.78 | 14.66552 |
| T42 | 31.86 | 30.32 | 1.54 | 34.22 | 32.58 | 1.64 | 20 | 16.27 | 3.73 | 13.48522 |
| T43 | 31.76 | 29.45 | 2.31 | 36.17 | 34.62 | 1.55 | 20 | 17.9 | 2.1 | [symbol] |
| T44 | 33.19 | 31.3 | 1.89 | 35.9 | 34.02 | 1.88 | 20 | 17.04 | 2.96 | 12.79209 |
| T45 | 34.17 | 32.73 | 1.44 | 34.3 | 32.4 | 1.9 | 20 | 15.63 | 4.37 | 15.27184 |
| T46 | 35.73 | 33.58 | 2.15 | 36.91 | 35.44 | 1.47 | 20 | 17.27 | 2.73 | [symbol] |
| T47 | 32.08 | 31.48 | 0.6 | 33.26 | 31.65 | 1.61 | 20 | 15.31 | 4.69 | 14.24312 |
| T48 | 31.63 | 30.43 | 1.2 | 34.05 | 32.13 | 1.92 | 20 | 14.93 | 5.07 | 16.50192 |
| T49 | 32.78 | 31.47 | 1.31 | 34.17 | 32.11 | 2.06 | 20 | 15.96 | 4.04 | 14.72287 |
| T50 | 33.7 | 32.26 | 1.44 | 34.25 | 33.01 | 1.24 | 20 | 15.52 | 4.48 | 14.13312 |
| T51 | 32.57 | 31.17 | 1.4 | 34.51 | 32.37 | 2.14 | 20 | 16.08 | 3.92 | 14.7506 |

TABLE 22
Test data of CRC samples

| | | | | | | | | | | Alu | | | |
|---|---|---|---|---|---|---|---|---|---|---|---|---|---|
| | SDC2 | | | WDR17 | | | ADHFE1 | | | Fixed | | | Weight |
| | ct1 | ct2 | Δct | ct1 | ct2 | Δct | ct1 | ct2 | Δct | value | ct | Δct | value |
| T1 | 28.05 | 27.21 | 0.84 | 36.16 | 32.93 | 3.23 | 34.7 | 32.13 | 2.57 | 20 | 15.69 | 4.31 | 15.63738 |
| T2 | 29.04 | 28.29 | 0.75 | 36.61 | 32.56 | 4.05 | 34.95 | 32.3 | 2.65 | 20 | 15.72 | 4.28 | 16.17201 |
| T3 | 29.63 | 28.64 | 0.99 | 38.34 | 33.73 | 4.61 | 35.83 | 34.03 | 1.8 | 20 | 17.09 | 2.91 | 14.43987 |
| T4 | 27.57 | 26.42 | 1.15 | 38.24 | 31.65 | 6.59 | 33.33 | 31.08 | 2.25 | 20 | 14.11 | 5.89 | 22.35338 |
| T5 | 29.46 | 28.93 | 0.53 | 37.15 | 33.98 | 3.17 | 35.95 | 34.5 | 1.45 | 20 | 17.01 | 2.99 | 11.28088 |
| T6 | 29.37 | 28.83 | 0.54 | 38.69 | 34.34 | 4.35 | 35.68 | 34.1 | 1.58 | 20 | 16.83 | 3.17 | 12.90573 |
| T7 | 27.21 | 25.39 | 1.82 | 33.08 | 28.76 | 4.32 | 30.82 | 29.03 | 1.79 | 20 | 20.39 | −0.39 | [symbol] |
| T8 | 28.69 | 27.76 | 0.93 | 35.5 | 31.75 | 3.75 | 33.08 | 31.46 | 1.62 | 20 | 14.78 | 5.22 | 17.0934 |
| T9 | 28.91 | 27.76 | 1.15 | 36.24 | 31.66 | 4.58 | 33.84 | 32.19 | 1.65 | 20 | 14.5 | 5.5 | 19.14965 |
| T10 | 28.02 | 27.2 | 0.82 | 38.1 | 33.2 | 4.9 | 35.96 | 33.91 | 2.05 | 20 | 17.13 | 2.87 | 14.32364 |
| T11 | 29.36 | 28.2 | 1.16 | 38.35 | 33.24 | 5.11 | 37.1 | 34.08 | 3.02 | 20 | 16.75 | 3.25 | 17.21281 |
| T12 | 29.45 | 28.72 | 0.73 | 36.72 | 33.47 | 3.25 | 34.94 | 32.52 | 2.42 | 20 | 16.06 | 3.94 | 14.52824 |
| T13 | 29.27 | 28.63 | 0.64 | 36.34 | 33.47 | 2.87 | 34.63 | 33.12 | 1.51 | 20 | 17.06 | 2.94 | [symbol] |
| T14 | 27.98 | 27.52 | 0.46 | 37.32 | 34.02 | 3.3 | 36.12 | 33.29 | 2.83 | 20 | 16.37 | 3.63 | 13.5359 |
| T15 | 26.08 | 25.03 | 1.05 | 33.11 | 28.12 | 4.99 | 29.89 | 28.76 | 1.13 | 20 | 20.59 | −0.59 | [symbol] |
| T16 | 29.04 | 28.01 | 1.03 | 38.5 | 34.19 | 4.31 | 34.89 | 33.65 | 1.24 | 20 | 16.24 | 3.76 | 15.17984 |
| T17 | 27.39 | 27.21 | 0.18 | 41.03 | 35.00 | 6.03 | 36.05 | 34.2 | 1.84 | 20 | 17.42 | 2.58 | 12.61608 |
| T18 | 27.48 | 25.7 | 1.78 | 33.25 | 30.14 | 3.11 | 34.15 | 32.71 | 1.44 | 20 | 16.02 | 3.98 | 17.05988 |
| T19 | 28.3 | 27.68 | 0.62 | 37.42 | 33.50 | 3.92 | 35.59 | 33.06 | 2.53 | 20 | 16.51 | 3.49 | 14.17102 |
| T20 | 26.96 | 24.31 | 2.65 | 34.85 | 29.07 | 5.78 | 29.47 | 27.6 | 1.87 | 20 | 12.1 | 7.9 | 29.5927 |
| T21 | 29.23 | 28.29 | 0.94 | 38.49 | 32.83 | 5.66 | 34.25 | 33.23 | 1.02 | 20 | 15.77 | 4.23 | 16.79777 |
| T22 | 29.12 | 28.14 | 0.98 | 37.44 | 33.13 | 4.31 | 34.44 | 31.15 | 3.29 | 20 | 15.12 | 4.88 | 18.80323 |
| T23 | 29.00 | 28.38 | 0.62 | 36.46 | 33.72 | 2.74 | 35.69 | 34.22 | 1.47 | 20 | 16.88 | 3.12 | [symbol] |
| T24 | 28.53 | 27.21 | 1.32 | 36.41 | 32.87 | 3.54 | 35.11 | 34.09 | 1.02 | 20 | 16.14 | 3.86 | 15.35148 |
| T25 | 29.37 | 28.34 | 1.03 | 35.49 | 32.77 | 2.72 | 35.25 | 33.03 | 2.22 | 20 | 15.99 | 4.01 | 14.93933 |
| T26 | 29.36 | 28.34 | 1.02 | 35.23 | 32.85 | 2.38 | 34.73 | 33.04 | 1.69 | 20 | 15.55 | 4.45 | 14.81262 |
| T27 | 29.24 | 28.16 | 1.08 | 36.48 | 32.86 | 3.62 | 33.96 | 31.9 | 2.06 | 20 | 15.64 | 4.36 | 16.4355 |
| T28 | 29.08 | 26.27 | 2.81 | 35.33 | 31.31 | 4.02 | 32.38 | 29.03 | 3.35 | 20 | 13.09 | 6.91 | 28.10692 |
| T29 | 28.13 | 26.19 | 1.94 | 36.14 | 31.23 | 4.91 | 31.84 | 29.23 | 2.61 | 20 | 13.34 | 6.66 | 24.96385 |
| T30 | 29.66 | 28.97 | 0.69 | 37.01 | 33.49 | 3.52 | 35.48 | 31.57 | 3.91 | 20 | 15.31 | 4.69 | 17.30946 |
| T31 | 28.69 | 27.35 | 1.34 | 35.58 | 31.73 | 3.85 | 32.65 | 31.34 | 1.31 | 20 | 14.39 | 5.61 | 18.9307 |
| T32 | 29.21 | 28.25 | 0.96 | 36.02 | 33.00 | 3.02 | 34.37 | 32.74 | 1.63 | 20 | 15.41 | 4.59 | 15.42402 |
| T33 | 28.63 | 28.05 | 0.58 | 35.66 | 32.17 | 3.49 | 34.27 | 32.3 | 1.97 | 20 | 15.06 | 4.94 | 15.52049 |
| T34 | 29.04 | 28.39 | 0.65 | 36.18 | 33.43 | 2.75 | 37.46 | 35.72 | 1.74 | 20 | 18.12 | 1.88 | [symbol] |
| T35 | 28.83 | 28.21 | 0.62 | 35.63 | 32.78 | 2.85 | 38.19 | 34.44 | 3.75 | 20 | 16.17 | 3.83 | 14.82016 |
| T36 | 28.29 | 27.7 | 0.59 | 34.78 | 31.6 | 3.18 | 33.76 | 31.71 | 2.05 | 20 | 15.28 | 4.72 | 14.95279 |
| T37 | 28.69 | 27.29 | 1.4 | 34.71 | 31.28 | 3.43 | 33.77 | 32.01 | 1.76 | 20 | 14.02 | 5.98 | 19.75624 |
| T38 | 28.71 | 25.89 | 2.82 | 35.49 | 30.67 | 4.82 | 32.44 | 29.45 | 2.99 | 20 | 12.22 | 7.78 | 30.05553 |
| T39 | 29.29 | 29.1 | 0.19 | 36.27 | 33.85 | 2.42 | 36.43 | 33.51 | 2.92 | 20 | 16.63 | 3.37 | [symbol] |
| T40 | 28.78 | 27.25 | 1.53 | 35.94 | 31.56 | 4.38 | 32.55 | 30.13 | 2.42 | 20 | 13.77 | 6.23 | 22.16727 |
| T41 | 29.23 | 28.4 | 0.83 | 35.73 | 33.20 | 2.53 | 34.47 | 32.8 | 1.67 | 20 | 16.67 | 3.33 | 12.42772 |
| T42 | 29.27 | 28.36 | 0.91 | 35.22 | 32.90 | 2.32 | 35.24 | 33.48 | 1.76 | 20 | 16.87 | 3.13 | 12.23639 |
| T43 | 28.98 | 27.72 | 1.26 | 34.93 | 31.99 | 2.94 | 34.22 | 33.79 | 0.43 | 20 | 15.99 | 4.01 | 14.25756 |
| T44 | 29.00 | 27.84 | 1.16 | 35.12 | 31.18 | 3.94 | 34.38 | 32.11 | 2.27 | 20 | 14.59 | 5.41 | 18.97708 |
| T45 | 28.52 | 27.75 | 0.77 | 35.62 | 31.39 | 4.23 | 34.07 | 32.89 | 1.18 | 20 | 14.64 | 5.36 | 16.85686 |
| T46 | 29.08 | 28.55 | 0.53 | 36.17 | 33.07 | 3.10 | 35.95 | 34 | 1.95 | 20 | 16.39 | 3.61 | 12.71782 |
| T47 | 31.42 | 30.22 | 1.2 | 36.42 | 33.1 | 3.32 | 34.21 | 33.47 | 0.74 | 20 | 16.47 | 3.53 | 13.91703 |
| T48 | 30.82 | 30.19 | 0.63 | 36.06 | 33.5 | 2.56 | 36.97 | 35.5 | 1.47 | 20 | 17.81 | 2.19 | [symbol] |
| T49 | 31.11 | 30.06 | 1.05 | 36.36 | 34.17 | 2.19 | 35.89 | 34.07 | 1.82 | 20 | 17.2 | 2.8 | [symbol] |
| T50 | 31.03 | 27.92 | 3.11 | 35.23 | 32.08 | 3.15 | 33.3 | 30.19 | 3.11 | 20 | 13.31 | 6.69 | 27.65776 |
| T51 | 30.12 | 29.33 | 0.79 | 35.13 | 32.9 | 2.23 | 36.72 | 33.55 | 3.17 | 20 | 17.22 | 2.78 | 12.47387 |
| T52 | 26.31 | 23.8 | 2.51 | 33.71 | 29.47 | 4.24 | 30.66 | 28.88 | 1.78 | 20 | 12.47 | 7.53 | 26.89465 |
| T53 | 30.01 | 29.16 | 0.85 | 35.26 | 33 | 2.26 | 39.4 | 33.3 | 6.1 | 20 | 17.14 | 2.86 | 15.57242 |
| T54 | 27.34 | 24.97 | 2.37 | 33.37 | 29.63 | 3.74 | 30.75 | 29.37 | 1.38 | 20 | 13.06 | 6.94 | 24.56802 |
| T55 | 30.79 | 30.04 | 0.75 | 36.17 | 31.83 | 4.34 | 35.43 | 35.39 | 0.04 | 20 | 16.34 | 3.66 | 12.98424 |
| T56 | 30.59 | 28.4 | 2.19 | 35.75 | 31.83 | 3.92 | 34.68 | 32.35 | 2.33 | 20 | 13.67 | 6.33 | 24.0066 |
| T57 | 27.87 | 26.91 | 0.96 | 33.6 | 29.51 | 4.09 | 32.02 | 30.18 | 1.84 | 20 | 12.38 | 7.62 | 21.76056 |

TABLE 23
Test data of healthy samples paired with CRC group

| | | | | | | | | | | | | | Weight |
|---|---|---|---|---|---|---|---|---|---|---|---|---|---|
| | SDC2 | | | WDR17 | | | ADHFE1 | | | Alu | | | value |
| N1 | 29.31 | 29.11 | 0.20 | 37.32 | 34.12 | 3.2 | 36.44 | 34.78 | 1.66 | 20 | 17.28 | 2.72 | 9.94822 |
| N2 | 27.69 | 27.48 | 0.21 | 37.27 | 34.37 | 2.9 | 34.7 | 35.24 | −0.54 | 20 | 17.9 | 2.1 | 6.59238 |
| N3 | 27.67 | 27.65 | 0.02 | 37.6 | 34.37 | 3.23 | 35.25 | 34.13 | 1.12 | 20 | 17.79 | 2.21 | 8.01583 |
| N4 | 28.9 | 28.5 | 0.4 | 37.39 | 33.77 | 3.62 | 36.14 | 35.21 | 0.93 | 20 | 17.12 | 2.88 | 10.62125 |
| N5 | 27.84 | 27.8 | 0.04 | 36.92 | 35.74 | 1.18 | 36.86 | 34.67 | 2.19 | 20 | 18.5 | 1.5 | 5.86097 |
| N6 | 27.94 | 27.73 | 0.21 | 30.83 | 29.03 | 1.8 | 36.23 | 34.41 | 1.82 | 20 | 17.55 | 2.45 | 8.29221 |
| N7 | 29.06 | 28.82 | 0.24 | 33.05 | 32.01 | 1.04 | 36.38 | 34.57 | 1.81 | 20 | 17.03 | 2.97 | 8.50302 |
| N8 | 28.9 | 28.25 | 0.65 | 33.79 | 32.98 | 0.81 | 36.33 | 34.28 | 2.05 | 20 | 16.84 | 3.16 | 10.19137 |
| N9 | 28.04 | 27.58 | 0.46 | 37.78 | 34.34 | 3.44 | 37.3 | 35.34 | 1.96 | 20 | 17.91 | 2.09 | 10.28021 |
| N10 | 28.02 | 27.66 | 0.36 | 36.96 | 33.16 | 3.8 | 35.68 | 35.07 | 0.61 | 20 | 17.36 | 2.64 | 9.96441 |
| N11 | 29.43 | 28.73 | 0.70 | 37.51 | 34.64 | 2.87 | 36.14 | 34.21 | 1.93 | 20 | 17.3 | 2.7 | 11.51489 |
| N12 | 29.21 | 28.77 | 0.44 | 35.76 | 33.14 | 2.62 | 37.52 | 34.31 | 3.21 | 20 | 17.04 | 2.96 | 12.02665 |
| N13 | 29.31 | 28.55 | 0.76 | 32.69 | 31.18 | 1.51 | 36.32 | 34.02 | 2.3 | 20 | 17.19 | 2.81 | 10.89917 |
| N14 | 28.86 | 28.7 | 0.16 | 37.54 | 34.09 | 3.45 | 34.45 | 34.9 | −0.45 | 20 | 17.63 | 2.37 | 7.50614 |
| N15 | 29.07 | 28.71 | 0.36 | 38.95 | 34.28 | 4.67 | 35.92 | 34.88 | 1.04 | 20 | 17.42 | 2.58 | 11.12628 |
| N16 | 28.13 | 27.88 | 0.25 | 38.77 | 34.36 | 4.41 | 37.19 | 34.16 | 3.03 | 20 | 17.65 | 2.35 | 11.97134 |
| N17 | 27.94 | 27.66 | 0.28 | 40.73 | 36.24 | 4.49 | 37.15 | 36.29 | 0.86 | 20 | 18.74 | 1.26 | 8.2985 |
| N18 | 29.03 | 28.86 | 0.17 | 38.59 | 34.98 | 3.61 | 37.16 | 36.05 | 1.11 | 20 | 17.62 | 2.38 | 9.17029 |
| N19 | 26.9 | 26.69 | 0.21 | 38.44 | 34.87 | 3.57 | 37.43 | 36.58 | 0.85 | 20 | 18.31 | 1.69 | 7.86498 |
| N20 | 27.95 | 27.68 | 0.27 | 37.72 | 34.43 | 3.29 | 36.21 | 35.19 | 1.02 | 20 | 17.17 | 2.83 | 9.85917 |
| N21 | 28.68 | 27.97 | 0.71 | 32.88 | 31.2 | 1.68 | 35.04 | 33.43 | 1.61 | 20 | 15.52 | 4.48 | [symbol] |
| N22 | 28.93 | 28.54 | 0.39 | 37.35 | 33.75 | 3.6 | 35.75 | 34.04 | 1.71 | 20 | 17.2 | 2.8 | 11.16153 |
| N23 | 29.00 | 28.59 | 0.41 | 37.83 | 34.09 | 3.74 | 35.78 | 34.07 | 1.71 | 20 | 16.96 | 3.04 | 11.76961 |
| N24 | 28.83 | 28.50 | 0.33 | 38.02 | 33.81 | 4.21 | 36.28 | 35.93 | 0.35 | 20 | 17.1 | 2.9 | 10.46325 |
| N25 | 28.58 | 28.25 | 0.33 | 34.17 | 31.94 | 2.23 | 34.56 | 33.12 | 1.44 | 20 | 16.68 | 3.32 | 10.22436 |
| N26 | 29.62 | 29.10 | 0.52 | 36.66 | 33.37 | 3.29 | 38.33 | 35.64 | 2.69 | 20 | 18.08 | 1.92 | 10.72871 |
| N27 | 29.68 | 29.12 | 0.56 | 36.20 | 33.93 | 2.27 | 36.81 | 35.08 | 1.73 | 20 | 18.04 | 1.96 | 9.02431 |
| N28 | 28.81 | 28.15 | 0.66 | 36.13 | 33.64 | 2.49 | 35.44 | 34.63 | 0.81 | 20 | 16.91 | 3.09 | 10.61376 |
| N29 | 29.53 | 29.10 | 0.43 | 34.71 | 32.85 | 1.86 | 35.48 | 35.5 | −0.02 | 20 | 17.62 | 2.38 | 7.2545 |
| N30 | 29.57 | 29.20 | 0.37 | 35.50 | 33.24 | 2.26 | 35.19 | 34.76 | 0.43 | 20 | 17.73 | 2.27 | 7.68488 |
| N31 | 29.53 | 29.42 | 0.11 | 35.24 | 32.44 | 2.80 | 37.54 | 36.56 | 0.98 | 20 | 18.18 | 1.82 | 7.10698 |
| N32 | 29.42 | 29.34 | 0.08 | 37.37 | 33.43 | 3.94 | 36.87 | 37.32 | −0.45 | 20 | 18.88 | 1.12 | 5.62699 |
| N33 | 29.01 | 28.4 | 0.61 | 35.44 | 33.37 | 2.07 | 35.28 | 35.49 | −0.21 | 20 | 18.12 | 1.88 | 7.04963 |
| N34 | 28.99 | 28.52 | 0.47 | 32.23 | 30.39 | 1.84 | 37.03 | 35.12 | 1.91 | 20 | 18.1 | 1.9 | 8.36443 |
| N35 | 28.68 | 28.2 | 0.48 | 36.03 | 32.66 | 3.37 | 34.28 | 34.04 | 0.24 | 20 | 16.51 | 3.49 | 11.02095 |
| N36 | 28.98 | 28.54 | 0.44 | 36.11 | 32.87 | 3.24 | 35.33 | 34.6 | 0.73 | 20 | 17.98 | 2.02 | 8.75023 |
| N37 | 29.12 | 28.49 | 0.63 | 36 | 33 | 3 | 35.14 | 33.57 | 1.57 | 20 | 16.87 | 3.13 | 11.79432 |
| N38 | 28.85 | 28.45 | 0.40 | 36.45 | 33.02 | 3.43 | 34.41 | 33.84 | 0.57 | 20 | 16.65 | 3.35 | 10.88546 |
| N39 | 29.36 | 28.67 | 0.69 | 35.81 | 32.78 | 3.03 | 35.75 | 34.87 | 0.88 | 20 | 18.15 | 1.85 | 9.23469 |
| N40 | 28.63 | 28.54 | 0.09 | 32.8 | 30.09 | 2.71 | 35.78 | 33.79 | 1.99 | 20 | 18.15 | 1.85 | 7.94352 |
| N41 | 29.38 | 28.75 | 0.63 | 35.91 | 33.08 | 2.83 | 34.91 | 34.17 | 0.74 | 20 | 16.42 | 3.58 | 11.60628 |
| N42 | 29.32 | 29.17 | 0.15 | 35.10 | 32.89 | 2.21 | 36.26 | 35.39 | 0.87 | 20 | 17.65 | 2.35 | 7.44306 |
| N43 | 29.57 | 29.21 | 0.36 | 35.78 | 32.74 | 3.04 | 35.09 | 36.02 | −0.93 | 20 | 17.33 | 2.67 | 7.8255 |
| N44 | 30.97 | 30.93 | 0.04 | 37.99 | 34.21 | 3.78 | 35.6 | 35.62 | −0.02 | 20 | 19.26 | 0.74 | 5.09852 |
| N45 | 31.27 | 30.07 | 1.2 | 36.84 | 33.4 | 3.44 | 36.22 | 34.55 | 1.67 | 20 | 16.86 | 3.14 | [symbol] |
| N46 | 30.93 | 30.44 | 0.49 | 37.02 | 34.39 | 2.63 | 36.35 | 35.28 | 1.07 | 20 | 18.17 | 1.83 | 8.31242 |
| N47 | 31.09 | 30.32 | 0.77 | 36.24 | 33.93 | 2.31 | 35.37 | 35.12 | 0.25 | 20 | 18.51 | 1.49 | 7.59838 |
| N48 | 30.23 | 29.52 | 0.71 | 35.75 | 32.94 | 2.81 | 37.61 | 35.27 | 2.34 | 20 | 17.88 | 2.12 | 10.89886 |
| N49 | 30.03 | 29.48 | 0.55 | 35.28 | 33.24 | 2.04 | 35.16 | 34.77 | 0.39 | 20 | 17.65 | 2.35 | 8.16692 |
| N50 | 29.93 | 28.86 | 1.07 | 35.22 | 32.52 | 2.7 | 36.65 | 35.04 | 1.61 | 20 | 17.05 | 2.95 | 12.70678 |
| N51 | 30.52 | 29.24 | 1.28 | 35.18 | 32.52 | 2.66 | 36.33 | 33.9 | 2.43 | 20 | 17.11 | 2.89 | [symbol] |
| N52 | 31.25 | 31.09 | 0.16 | 36.36 | 34.99 | 1.37 | 37.94 | 36.41 | 1.53 | 20 | 17.63 | 2.37 | 7.29428 |
| N53 | 30.91 | 30.74 | 0.17 | 37.54 | 33.87 | 3.67 | 37.85 | 34.64 | 3.21 | 20 | 16.99 | 3.01 | 12.2455 |
| N54 | 31.24 | 30.89 | 0.35 | 38.97 | 35.04 | 3.93 | 37.79 | 37.79 | 0 | 20 | 18.14 | 1.86 | 8.18242 |

1-14. canceled

15. A kit for detecting DNA methylation based on quantitative polymerase chain reaction (qPCR), comprising parameter settings for PCR with two programs, which denature conditions are conducted in high temperature and low temperature respectively, wherein the ΔCt obtained by subtracting of the Ct value of qPCR conducted in two programs with same reaction mixture, and the following components: a Taq enzyme premix system, primers and probes of target genes for qPCR , and reagents for plotting a reference curve.

16. The kit according to claim 15, (1) wherein the reagents for plotting the reference curve comprise deoxynucleotide triphosphate (dNTP) reagents with different proportions of methylated cytosine and a common reagent for polymerase chain reaction (PCR);
(2) wherein the target genes for detection of lung cancer or CRC comprises, but is not limited to, one or more selected from the group consisting of the following genes: APC, SHOX2, Apobec3B, P53, AID, HOXD12, Alu, SDC2, WDR17, and ADHFE1.

17. The kit according to claim 16, wherein the dNTP reagents with different proportions of methylated cytosine each comprise equal molar concentrations of deoxyadenosine triphosphate (dATP), deoxythymidine triphosphate (dTTP), deoxyguanosine triphosphate (dGTP), and a reagent A; and
the reagent A is 5′-methyl-deoxycytidine triphosphate (5′-m-dCTP) and/or deoxycytidine triphosphate (dCTP).

18. The kit according to claim 17, wherein in the reagent A, a molar ratio of the 5′-m-dCTP to the dCTP falls into the parameters as follows: 1:0, 1:(1-30), or 0:1.

19. A method for detecting DNA methylation, comprising the following steps:
1. using the primers and probes in the kit according to claim 15 to prepare a reaction system for qPCR detection, and conducting high-temperature denaturation amplification and low-temperature denaturation amplification to obtain Ct_{X-HT} for the high-temperature denaturation amplification and Ct_{X-LH} for the low-temperature denaturation amplification;
2) substituting Ct_{X-LH} in and Ct_{X-HT} obtained in step 1) into equation I to obtain ΔCtx of a target gene,
ΔCtx=Ct_{X-LH}−Ct_{X-HT} equation I
wherein Ct_{X-LH} represents a Ct value of an X gene when amplified under low-temperature denaturation, Ct_{X-HT} represents a Ct value of an X gene when amplified under high-temperature denaturation, and X represents a target gene;
3) using the reagents for plotting the reference curve in the kit according to claim 15 to amplify fragments of different DNA methylation levels for the target gene, subjecting resulting PCR data to the operations in step 1) and step 2) to obtain ΔCtx′ of the fragments of different DNA methylation levels, and based on a logarithm relationship between different proportions of methylated cytosine and ΔCtx′, plotting the reference curve; and
4) comparing ACtx of the target gene in step 2) with ΔCtx′ in the reference curve in step 3) to obtain a proportion of methylated cytosine in the target gene, such as to determine a DNA methylation level of the target gene.

20. The method according to claim 19, wherein the target gene comprises, but is not limited to, one or more selected from the group consisting of the following genes: APC, SHOX2, Apobec3B, P53, AID, HOXD12, Alu, SDC2, WDR17, and ADHFE1.

21. The method according to claim 19, wherein reaction conditions for the high-temperature denaturation amplification are as follows: predenaturation at 95° C. for 5 min; and 94° C. for 5 s, 60° C. to 62° C. for 15 s, and 72° C. for 30 s, with 45 cycles; and
reaction conditions for the low-temperature denaturation amplification are as follows: 85° C. to 88° C. for 15 s, 60° C. to 62° C. for 15 s, and 72° C. for 30 s, with 18 cycles; and 94° C. for 5 s, 60° C. to 62° C. for 15 s, and 72° C. for 30 s, with 27 cycles.

22. The method according to claim 20, wherein reaction conditions for the high-temperature denaturation amplification are as follows: predenaturation at 95° C. for 5 min; and 94° C. for 5 s, 60° C. to 62° C. for 15 s, and 72° C. for 30 s, with 45 cycles; and
reaction conditions for the low-temperature denaturation amplification are as follows: 85° C. to 88° C. for 15 s, 60° C. to 62° C. for 15 s, and 72° C. for 30 s, with 18 cycles; and 94° C. for 5 s, 60° C. to 62° C. for 15 s, and 72° C. for 30 s, with 27 cycles.

23. A method for distinguishing a DNA methylation change of one study population from a DNA methylation change of the other study population by using the kit according to claim 15.

24. The method according to claim 23, wherein the target gene comprises, but is not limited to, one or more selected from the group consisting of the following genes: APC, SHOX2, Apobec3B, P53, AID, HOXD12, Alu, SDC2, WDR17, and ADHFE1.

25. The method according to claim 23, wherein reaction conditions for the high-temperature denaturation amplification are as follows: predenaturation at 95° C. for 5 min; and 94° C. for 5 s, 60° C. to 62° C. for 15 s, and 72° C. for 30 s, with 45 cycles; and
reaction conditions for the low-temperature denaturation amplification are as follows: 85° C. to 88° C. for 15 s, 60° C. to 62° C. for 15 s, and 72° C. for 30 s, with 18 cycles; and 94° C. for 5 s, 60° C. to 62° C. for 15 s, and 72° C. for 30 s, with 27 cycles.

26. The method according to claim 24, wherein reaction conditions for the high-temperature denaturation amplification are as follows: predenaturation at 95° C. for 5 min; and 94° C. for 5 s, 60° C. to 62° C. for 15 s, and 72° C. for 30 s, with 45 cycles; and
reaction conditions for the low-temperature denaturation amplification are as follows: 85° C. to 88° C. for 15 s, 60° C. to 62° C. for 15 s, and 72° C. for 30 s, with 18 cycles; and 94° C. for 5 s, 60° C. to 62° C. for 15 s, and 72° C. for 30 s, with 27 cycles.

27. The method according to claim 23, wherein when the target gene comprises two or more target genes, a method for distinguishing a DNA methylation change of one study population from a DNA methylation change of the other study population comprises the following steps:
after ΔCt of each target gene is obtained, subjecting ΔCt of a target gene corresponding to each of samples of a population 1 and samples of a population 2 to statistical regression analysis to obtain a threshold and a weight value equation II for joint detection of target genes, wherein ΔCt of each target gene is substituted into the weight value equation II to calculate a weight value for joint detection of multiple target genes of each sample,
weight value for joint detection of multiple target genes=a1×ΔCtₓ₁+a2×ΔCtₓ₂ +. . . +an×ΔCtₓₙ equation II
wherein “a1”, “a2”, and “an” each represent a corresponding coefficient obtained during statistical analysis of each target gene;
qualitative comparison between the two populations: comparing a weight value of each of the samples in the population 1 and the samples in the population 2 with a threshold obtained by binary logistic regression analysis, wherein a sample with a weight value lower the threshold is a lowly methylated sample and is defined as negative, and a sample with a weight value higher than the threshold is a highly methylated sample and is defined as positive; and determining a difference between the two populations through statistical analysis; and
differential determination between the two populations: calculating a mean value of weight values of the samples in the population 1 and a mean value of weight values of the samples in the population 2, and conducting T test analysis, wherein when the mean value of the weight values of the samples in the population 1 is significantly different from the mean value of the weight values of the samples in the population 2, it indicates that there is a significant DNA methylation difference between the two populations.

28. The method according to claim 23, wherein the two study populations are a healthy population and a lung cancer patient population.

29. The method according to claim 24, wherein the two study populations are a healthy population and a lung cancer patient population.

30. The method according to claim 23, wherein the two study populations are a healthy population and a colorectal cancer (CRC) patient population.

31. The method according to claim 24, wherein the two study populations are a healthy population and a colorectal cancer (CRC) patient population.


SEQUENCE LISTING (40 sequences)

SEQ ID NO: 1 - DNA, 21 bp, synthetic construct
  source 1..21: mol_type = other DNA
atcttgtgctaatccttctgc

SEQ ID NO: 2 - DNA, 17 bp, synthetic construct
  source 1..17: mol_type = other DNA
ccgcacagcctgcctag

SEQ ID NO: 3 - DNA, 21 bp, synthetic construct
  source 1..21: mol_type = other DNA
ctgcggacctcccccgactct

SEQ ID NO: 4 - DNA, 19 bp, synthetic construct
  source 1..19: mol_type = other DNA
agttgcgaggggaggtaat

SEQ ID NO: 5 - DNA, 19 bp, synthetic construct
  source 1..19: mol_type = other DNA
gggaaatgggaactgatgc

SEQ ID NO: 6 - DNA, 22 bp, synthetic construct
  source 1..22: mol_type = other DNA
ccctgccggagcgcgcttgttc

SEQ ID NO: 7 - DNA, 23 bp, synthetic construct
  source 1..23: mol_type = other DNA
gccagagccagagaaacatgaag

SEQ ID NO: 8 - DNA, 23 bp, synthetic construct
  source 1..23: mol_type = other DNA
tgcttacagcgtccttgcagttg

SEQ ID NO: 9 - DNA, 21 bp, synthetic construct
  source 1..21: mol_type = other DNA
ccggggcctcccacaccaatg

SEQ ID NO: 10 - DNA, 22 bp, synthetic construct
  source 1..22: mol_type = other DNA
cacttgtgccctgactttcaac

SEQ ID NO: 11 - DNA, 21 bp, synthetic construct
  source 1..21: mol_type = other DNA
gtcatgtgctgtgactgcttg

SEQ ID NO: 12 - DNA, 24 bp, synthetic construct
  source 1..24: mol_type = other DNA
agatggccatggcgcggacgcggg

SEQ ID NO: 13 - DNA, 23 bp, synthetic construct
  source 1..23: mol_type = other DNA
ttgaagtgtctactgttactgcc

SEQ ID NO: 14 - DNA, 22 bp, synthetic construct
  source 1..22: mol_type = other DNA
ctgtcataggcagagtcacaca

SEQ ID NO: 15 - DNA, 24 bp, synthetic construct
  source 1..24: mol_type = other DNA
ctagctatggagcatggactgggc

SEQ ID NO: 16 - DNA, 19 bp, synthetic construct
  source 1..19: mol_type = other DNA
cagcctcgtccttcgccat

SEQ ID NO: 17 - DNA, 23 bp, synthetic construct
  source 1..23: mol_type = other DNA
ataagaggacaggctcagacgat

SEQ ID NO: 18 - DNA, 27 bp, synthetic construct
  source 1..27: mol_type = other DNA
cagcaccgcctacatccccccgacccg

SEQ ID NO: 19 - DNA, 19 bp, synthetic construct
  source 1..19: mol_type = other DNA
gtggctcacgcctgtaatc

SEQ ID NO: 20 - DNA, 20 bp, synthetic construct
  source 1..20: mol_type = other DNA
ttagtagagacggggtttca

SEQ ID NO: 21 - DNA, 23 bp, synthetic construct
  source 1..23: mol_type = other DNA
ttgggaggccgaggcgggcggat

SEQ ID NO: 22 - DNA, 19 bp, synthetic construct
  source 1..19: mol_type = other DNA
agggtcacgagccacttcc

SEQ ID NO: 23 - DNA, 18 bp, synthetic construct
  source 1..18: mol_type = other DNA
ggagccacgatggaaggc

SEQ ID NO: 24 - DNA, 23 bp, synthetic construct
  source 1..23: mol_type = other DNA
cctctcgggcgctccaatcagcg

SEQ ID NO: 25 - DNA, 20 bp, synthetic construct
  source 1..20: mol_type = other DNA
tcggcgtgtaatcctgtagg

SEQ ID NO: 26 - DNA, 18 bp, synthetic construct
  source 1..18: mol_type = other DNA
ccctcctgagcctgcttc

SEQ ID NO: 27 - DNA, 22 bp, synthetic construct
  source 1..22: mol_type = other DNA
cgggctcccctgggcgactggg

SEQ ID NO: 28 - DNA, 21 bp, synthetic construct
  source 1..21: mol_type = other DNA
agatttaaggcagaacccgag

SEQ ID NO: 29 - DNA, 21 bp, synthetic construct
  source 1..21: mol_type = other DNA
gtcacctgtctaagcctcaag

SEQ ID NO: 30 - DNA, 25 bp, synthetic construct
  source 1..25: mol_type = other DNA
cagtcgggctcagggccattttccc

SEQ ID NO: 31 - DNA, 148 bp, unidentified
  source 1..148: mol_type = other DNA
atcttgtgctaatccttctgccctgcggacctcccccgactctttactatgcgtgtcaactgccatcaacttccttgcttgctggggactggggccgcgagggcatacccccgaggggtacggggctagggctaggcaggctgtgcgg

SEQ ID NO: 32 - DNA, 174 bp, unidentified
  source 1..174: mol_type = other DNA
agttgcgaggggaggtaatttactcaaaacaagaggccgccgttaggaagtataaatagtgggacttcaagcgcagcgtcgctatcagatctgaactctccgcaggaagaattgtcaaagatcttaacattgaacaagcgcgctccggcagggaagcatcagttcccatttccc

SEQ ID NO: 33 - DNA, 138 bp, unidentified
  source 1..138: mol_type = other DNA
gccagagccagagaaacatgaagcaccccggggcctcccacaccaatgcctgagcaggaatggggaggggccatgactcataaggccctgggaggtcactttaaggagggctgtccaactgcaaggacgctgtaagca

SEQ ID NO: 34 - DNA, 180 bp, unidentified
  source 1..180: mol_type = other DNA
cacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgac

SEQ ID NO: 35 - DNA, 145 bp, unidentified
  source 1..145: mol_type = other DNA
ttgaagtgtctactgttactgcctcctgatctttgctagctatggagcatggactgggcttttagagcagcagccccaaaggaacctaaacattaaagcagagctgccctcaatggtttaacctgtgtgactctgcctatgacag

SEQ ID NO: 36 - DNA, 155 bp, unidentified
  source 1..155: mol_type = other DNA
cagcctcgtccttcgccatcgaacattgcgggtgttatcataatactctgaagggggggaaaacgggtcggggggatgtaggcggtgctgaaatgaccggctttgaagaacctgcaggcaaagtttcgtccaatcgtctgagcctgtcctcttat

SEQ ID NO: 37 - DNA, 112 bp, unidentified
  source 1..112: mol_type = other DNA
gtggctcacgcctgtaatcccagcactttgggaggccgaggcgggcggatcacctgaggtcaggagttcgagaccagcctggccaacatggtgaaaccccgtctctactaaa

SEQ ID NO: 38 - DNA, 124 bp, synthetic construct
  source 1..124: mol_type = other DNA
agggtcacgagccacttccgcctccccctctcgggcgctccaatcagcgacctgtgtagctgtcacgtgggcctctggggcagaccctggagatcgggctcgcggcgccttccatcgtggctcc

SEQ ID NO: 39 - DNA, 107 bp, unidentified
  source 1..107: mol_type = other DNA
tcggcgtgtaatcctgtaggaatttctcccgggtttatctgggagtcacactgccgcctcctctccccagtcgcccaggggagcccggagaagcaggctcaggaggg

SEQ ID NO: 40 - DNA, 157 bp, unidentified
  source 1..157: mol_type = other DNA
agatttaaggcagaacccgaggcctacggagcagttaccttctacggcaatttcaagggtggatggtgcgagcgccgctggggcagctggcgttctggttcttactccgtgggaaaatggccctgagcccgactggcttgaggcttagacaggtgac